SEQUENCE LISTING (9 sequences)

SEQ ID NO: 1 - protein, 1276 aa, synthetic construct
MDWTWILFLVAAATRVHSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3834 bp, synthetic construct
  source 1..3834: mol_type = other DNA
atggattggacctggattctctttcttgtggccgcagcaacccgagtgcactcaagccagtgtgtgaacctgaccacaagaactcagctgcctcctgcctacaccaacagcttcaccagaggagtctactacccagacaaggtgttcagatcttctgtgctgcacagcacccaggacctcttcctgcccttcttcagcaacgtgacctggttccacgtgatctctggcaccaacggcaccaagagatttgacaaccctgtgctgccttttaatgatggtgtgtactttgccagcatcgagaagagcaacatcatccgaggctggatctttggcaccaccctggacagcaagacccagagcctgctgatcgtgaacaatgccaccaacgtggtcatcaaggtgtgtgagttccagttctgcaatgaccctttcctggaccacaagaacaacaagtcctggatggaatctgaatttagagtgtacagctctgccaacaactgcacatttgaatatgtgtcccagcctttcctgatggacctggagggcaaacaaggcaacttcaagaacctgagagaatttgtgttcaagaacattgatggctacttcaagatctacagcaagcacacacccatcatcgtgagagaacctgaagatcttcctcagggcttcagcgccctggagcctctggtggacctgcccatcggcatcaacatcaccaggttccagaccctgctggccctgcacagaagctacctgacacctggagacagcagcagcggctggactgctggagctgctgcctactacgtgggctacctgcagccaagaaccttcctgctgaagtacaacgaaaatggcaccatcacagatgctgttgactgtgccctggaccctctttctgagaccaagtgcaccctgaagtccttcacagtggagaagggcatctaccagaccagcaactttagagtgcagccaacagagagcatcgtgagatttccaaacatcaccaacctgtgcccatttgatgaagtgttcaatgccacaagattcgcctctgtgtacgcctggaacagaaaaagaatcagcaactgtgtggctgactactctgtgctgtacaacctggcccctttcttcaccttcaagtgctacggtgtgtctccaaccaagctgaatgacctgtgcttcaccaacgtgtatgctgacagctttgtcatcagaggagatgaagtgagacaaattgctcctggccagacaggcaacattgctgactacaactacaaacttcctgatgacttcacaggctgtgtcatcgcctggaacagcaacaagctggacagcaaagtttctggcaattacaactacctgtacagactcttcagaaaaagcaacctgaagccatttgaaagagacatcagcacagagatctaccaggctggcaacaagccctgcaacggagtggctggcttcaactgctacttccctctgcgctcctacagcttccggcccacctacggcgtgggccaccagccttacagagtggtggtgctgtcctttgagctgctgcacgcccctgccacagtgtgtggccccaagaagagcaccaacctggtgaagaacaaatgtgtgaacttcaactttaatggcctgaagggcacaggagtgctgacagagagcaacaagaagttcctgcccttccagcagtttggaagagacattgctgacaccacagatgctgtgagagatcctcagaccctggagatcctggacatcacaccttgctcctttggaggagtttctgtcatcacacctggcaccaacaccagcaaccaggtggctgtgctgtaccaaggagtcaactgcacagaagttcctgtggccatccatgctgaccagctgaccccaacatggagagtgtacagcacaggcagcaacgtgttccagacaagagctggctgcctgattggagctgagtacgtgaacaacagctatgaatgtgacatccctattggagctggcatctgtgcctcctaccagacccaaaccaagagccacagaagagcaagatctgtggccagccagagcatcatcgcctacaccatgagcctgggagctgaaaattctgtggcctacagcaataacagcatcgccatccccaccaacttcaccatctctgtgaccacagagatcctgcctgtgtccatgacaaaaacatctgtggactgcaccatgtacatctgtggagacagcacagaatgcagcaacctgctgctgcagtacggctccttctgcacccagctgaagagagccctgacaggaattgctgtggagcaggacaagaacacacaggaagtttttgcccaggtgaagcagatctacaaaacaccacccatcaagtactttggcggcttcaacttctcccagatcctgcctgacccttccaagcccagcaaaagaagcttcatcgaggacctgctgttcaacaaggtgaccctggctgatgctggcttcatcaagcagtatggagactgcctgggagacattgctgcccgggacctgatctgtgcccagaagttcaagggcctgacagtgctgcctcctctgctgacagatgaaatgatcgcccagtacacatctgccctgctggctggcaccatcaccagcggctggacatttggagctggagctgccctgcagatcccttttgccatgcagatggcctacagatttaatggcatcggcgtcacccagaacgtgctgtatgaaaaccagaagctgatcgccaaccagtttaacagtgccatcggcaagatccaggacagcctgagcagcacagcctctgccctgggcaagctgcaggatgtggtgaatcacaatgcccaagccctgaacaccctggtgaagcagctgagcagcaaatttggagccatctccagcgtgctgaatgacatcttctccaggctggaccctcctgaagctgaggtgcagatcgaccgcctgatcacaggccggctgcagagcctgcagacctacgtgacccagcagctgatcagagctgctgagatcagagcttctgccaacctggctgccaccaagatgtctgaatgtgtgctgggccagagcaaaagagtggacttctgtggcaagggctaccacctgatgtccttccctcagtctgctcctcacggcgtggtgttcctgcacgtgacctacgtgcctgcccaggagaagaacttcaccacagcccctgccatctgccacgacggcaaggcccacttcccaagagaaggcgtctttgtgtccaatggcacccactggttcgtgacacagagaaacttctacgagcctcagatcatcaccacagacaacacatttgtttctggcaactgtgatgtggtcatcggcatcgtgaacaacacagtttatgaccctctgcagcctgagctggacagcttcaaggaggagctggacaagtacttcaagaaccacaccagccctgatgtggacctgggcgacatctctggcatcaatgcctctgtggtgaacatccagaaggagatcgacaggctgaacgaagtggccaagaatctgaacgaaagcctcatcgacctgcaggagctgggcaagtacgagcagtacatcaagtggccttggtacatctggctgggcttcatcgccggcctcatcgccatcgtgatggtgaccatcatgctgtgctgcatgaccagctgctgctcttgcctgaagggctgctgcagctgtggcagctgctgcaagtttgatgaagatgattctgagcctgtgctgaagggcgtgaagctgcactacacatgataa

SEQ ID NO: 3 - DNA, 6788 bp, synthetic construct
  source 1..6788: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacctggattctctttcttgtggccgcagcaacccgagtgcactcaagccagtgtgtgaacctgaccacaagaactcagctgcctcctgcctacaccaacagcttcaccagaggagtctactacccagacaaggtgttcagatcttctgtgctgcacagcacccaggacctcttcctgcccttcttcagcaacgtgacctggttccacgtgatctctggcaccaacggcaccaagagatttgacaaccctgtgctgccttttaatgatggtgtgtactttgccagcatcgagaagagcaacatcatccgaggctggatctttggcaccaccctggacagcaagacccagagcctgctgatcgtgaacaatgccaccaacgtggtcatcaaggtgtgtgagttccagttctgcaatgaccctttcctggaccacaagaacaacaagtcctggatggaatctgaatttagagtgtacagctctgccaacaactgcacatttgaatatgtgtcccagcctttcctgatggacctggagggcaaacaaggcaacttcaagaacctgagagaatttgtgttcaagaacattgatggctacttcaagatctacagcaagcacacacccatcatcgtgagagaacctgaagatcttcctcagggcttcagcgccctggagcctctggtggacctgcccatcggcatcaacatcaccaggttccagaccctgctggccctgcacagaagctacctgacacctggagacagcagcagcggctggactgctggagctgctgcctactacgtgggctacctgcagccaagaaccttcctgctgaagtacaacgaaaatggcaccatcacagatgctgttgactgtgccctggaccctctttctgagaccaagtgcaccctgaagtccttcacagtggagaagggcatctaccagaccagcaactttagagtgcagccaacagagagcatcgtgagatttccaaacatcaccaacctgtgcccatttgatgaagtgttcaatgccacaagattcgcctctgtgtacgcctggaacagaaaaagaatcagcaactgtgtggctgactactctgtgctgtacaacctggcccctttcttcaccttcaagtgctacggtgtgtctccaaccaagctgaatgacctgtgcttcaccaacgtgtatgctgacagctttgtcatcagaggagatgaagtgagacaaattgctcctggccagacaggcaacattgctgactacaactacaaacttcctgatgacttcacaggctgtgtcatcgcctggaacagcaacaagctggacagcaaagtttctggcaattacaactacctgtacagactcttcagaaaaagcaacctgaagccatttgaaagagacatcagcacagagatctaccaggctggcaacaagccctgcaacggagtggctggcttcaactgctacttccctctgcgctcctacagcttccggcccacctacggcgtgggccaccagccttacagagtggtggtgctgtcctttgagctgctgcacgcccctgccacagtgtgtggccccaagaagagcaccaacctggtgaagaacaaatgtgtgaacttcaactttaatggcctgaagggcacaggagtgctgacagagagcaacaagaagttcctgcccttccagcagtttggaagagacattgctgacaccacagatgctgtgagagatcctcagaccctggagatcctggacatcacaccttgctcctttggaggagtttctgtcatcacacctggcaccaacaccagcaaccaggtggctgtgctgtaccaaggagtcaactgcacagaagttcctgtggccatccatgctgaccagctgaccccaacatggagagtgtacagcacaggcagcaacgtgttccagacaagagctggctgcctgattggagctgagtacgtgaacaacagctatgaatgtgacatccctattggagctggcatctgtgcctcctaccagacccaaaccaagagccacagaagagcaagatctgtggccagccagagcatcatcgcctacaccatgagcctgggagctgaaaattctgtggcctacagcaataacagcatcgccatccccaccaacttcaccatctctgtgaccacagagatcctgcctgtgtccatgacaaaaacatctgtggactgcaccatgtacatctgtggagacagcacagaatgcagcaacctgctgctgcagtacggctccttctgcacccagctgaagagagccctgacaggaattgctgtggagcaggacaagaacacacaggaagtttttgcccaggtgaagcagatctacaaaacaccacccatcaagtactttggcggcttcaacttctcccagatcctgcctgacccttccaagcccagcaaaagaagcttcatcgaggacctgctgttcaacaaggtgaccctggctgatgctggcttcatcaagcagtatggagactgcctgggagacattgctgcccgggacctgatctgtgcccagaagttcaagggcctgacagtgctgcctcctctgctgacagatgaaatgatcgcccagtacacatctgccctgctggctggcaccatcaccagcggctggacatttggagctggagctgccctgcagatcccttttgccatgcagatggcctacagatttaatggcatcggcgtcacccagaacgtgctgtatgaaaaccagaagctgatcgccaaccagtttaacagtgccatcggcaagatccaggacagcctgagcagcacagcctctgccctgggcaagctgcaggatgtggtgaatcacaatgcccaagccctgaacaccctggtgaagcagctgagcagcaaatttggagccatctccagcgtgctgaatgacatcttctccaggctggaccctcctgaagctgaggtgcagatcgaccgcctgatcacaggccggctgcagagcctgcagacctacgtgacccagcagctgatcagagctgctgagatcagagcttctgccaacctggctgccaccaagatgtctgaatgtgtgctgggccagagcaaaagagtggacttctgtggcaagggctaccacctgatgtccttccctcagtctgctcctcacggcgtggtgttcctgcacgtgacctacgtgcctgcccaggagaagaacttcaccacagcccctgccatctgccacgacggcaaggcccacttcccaagagaaggcgtctttgtgtccaatggcacccactggttcgtgacacagagaaacttctacgagcctcagatcatcaccacagacaacacatttgtttctggcaactgtgatgtggtcatcggcatcgtgaacaacacagtttatgaccctctgcagcctgagctggacagcttcaaggaggagctggacaagtacttcaagaaccacaccagccctgatgtggacctgggcgacatctctggcatcaatgcctctgtggtgaacatccagaaggagatcgacaggctgaacgaagtggccaagaatctgaacgaaagcctcatcgacctgcaggagctgggcaagtacgagcagtacatcaagtggccttggtacatctggctgggcttcatcgccggcctcatcgccatcgtgatggtgaccatcatgctgtgctgcatgaccagctgctgctcttgcctgaagggctgctgcagctgtggcagctgctgcaagtttgatgaagatgattctgagcctgtgctgaagggcgtgaagctgcactacacatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 4 - protein, 1279 aa, synthetic construct
MDWTWILFLVAAATRVHSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3837 bp, synthetic construct
  source 1..3837: mol_type = other DNA
atggattggacttggattctctttctcgttgctgcagccacacgcgttcatagcagccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacaca

SEQ ID NO: 6 - DNA, 6797 bp, synthetic construct
  source 1..6797: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctctttctcgttgctgcagccacacgcgttcatagcagccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 7 - protein, 1277 aa, synthetic construct
MDWTWILFLVAAATRVHSSQCVNFTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - DNA, 3837 bp, synthetic construct
  source 1..3837: mol_type = other DNA
atggattggacttggattctctttctcgttgctgcagccacacgcgttcatagcagccagtgtgtgaacttcaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatctctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgtgctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaacattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtgaagggcttcaactgctacttccctctgcagagctacggcttccagccaacatatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaaggagtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagccacagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggaccctccagaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataa

SEQ ID NO: 9 - DNA, 6791 bp, synthetic construct
  source 1..6791: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctctttctcgttgctgcagccacacgcgttcatagcagccagtgtgtgaacttcaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatctctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgtgctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaacattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtgaagggcttcaactgctacttccctctgcagagctacggcttccagccaacatatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaaggagtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagccacagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggaccctccagaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

(19) United States
(12) Patent Application Publication
(10) Pub. No.: US 2023/0340033 A1
(43) Pub. Date: Oct. 26, 2023

(54) Immunogenic Compositions Against SARS-COV-2 Variants And Their Methods Of Use

(71) Applicant: Inovio Pharmaceuticals, Inc., Plymouth Meeting, PA (US)

(72) Inventors: Kate Broderick, San Diego, CA (US); Trevor Smith, San Diego, CA (US); Richa Kalia, San Diego, CA (US); Viviane Machado de Mello Andrade, San Diego, CA (US)

(21) Appl. No.: 18/300,973

(22) Filed: Apr. 14, 2023

Related U.S. Application Data
(60) Provisional application No. 63/332,982, filed on Apr. 20, 2022.

(51) Int. Cl. C12N 15/63; A61P 37/04; C07K 14/165; A61K 45/06

(52) U.S. Cl. CPC C07K 14/165; A61K 45/06; A61P 37/04; C12N 15/63

(57) ABSTRACT

Disclosed herein are nucleic acid molecules encoding a Severe Acute Respiratory Syndrome coronavirus 2 (SARS-CoV-2) spike antigen, SARS-CoV-2 spike antigens, immunogenic compositions, and vaccines and their use in inducing immune responses and protecting against or treating a SARS-CoV-2 infection in a subject.

### CROSS-REFERENCE TO RELATED APPLICATIONS

This application claims the benefit of U.S. Provisional Appl. No. 63/332,982, filed Apr. 20, 2022, the entire contents of which are incorporated herein by reference.

### SEQUENCE LISTING

The contents of the electronic sequence listing (104409000846_Sequence Listing.xml; Size: 44,633 bytes; and Date of Creation: Apr. 3, 2023) is herein incorporated by reference in its entirety.

### TECHNICAL FIELD

The present invention relates to vaccines for Severe Acute Respiratory Syndrome coronavirus 2 (SARS-CoV-2) and methods of administering the vaccines.

### BACKGROUND

Coronavirus Disease-19 (COVID-19) remains a global pandemic. To date, SARS-CoV-2 has infected over 500 million people and over 6 million people have succumbed to disease [World Health Organization. WHO Coronavirus (COVID-19) Dashboard. 2022]; Available from: https_covid19_who_int]. Concerningly, Severe Acute Respiratory Syndrome coronavirus 2 (SARS-CoV-2) variants containing novel mutations impacting virological and epidemiological characteristics are driving an increased level of COVID-19 morbidity and mortality in many parts of the world.

Several variants have become a focus of attention. The B.1.1.7 (United Kingdom; Alpha), B.1.351 (South African; Beta)), P.1 (Brazilian; Gamma); B.1.617.2 (Delta); and B.1.1.529 (Omicron) variants have rapidly become dominant in some regions. Importantly, some of the mutations associated with these VOCs enhance resistance to neutralizing antibodies induced after infection or vaccination. [Collier, D.A., et al., Sensitivity of SARS-CoV-2 B.1.1.7 to mRNA vaccine-elicited antibodies. Nature, 2021; Garcia-Beltran, W.F., et al., Multiple SARS-CoV-2 variants escape neutralization by vaccine-induced humoral immunity. Cell, 2021; Wang, P., et al., Increased resistance of SARS-CoV-2 variant P.1 to Antibody Neutralization. Cell Host Microbe, 2021; Wang, Z., et al., mRNA vaccine-elicited antibodies to SARS-CoV-2 and circulating variants. Nature, 2021. 592(7855): p. 616-622; Wibmer, C.K., et al., SARS-CoV-2 501Y.V2 escapes neutralization by South African COVID-19 donor plasma. Nat Med, 2021. 27(4): p. 622-625; Liu, J., et al., BNT162b2-elicited neutralization of B.1.617 and other SARS-CoV-2 variants. Nature, 2021.].

New vaccine candidates that address the challenge of current and emerging SARS-CoV-2 variants are needed.

### SUMMARY

Provided herein are nucleic acid molecules encoding a SARS-CoV-2 spike antigen. According to some embodiments, the encoded SARS-CoV-2 spike antigen is a consensus antigen. In some embodiments, the nucleic acid molecule comprises: the nucleic acid sequence of nucleotides 55 to 3828 of SEQ ID NO: 2; the nucleic acid sequence of SEQ ID NO: 2; or the nucleic acid sequence of SEQ ID NO: 3. Also provided herein are nucleic acid molecules encoding a SARS-CoV-2 spike antigen, wherein the SARS-CoV-2 spike antigen comprises: the amino acid sequence set forth in residues 19 to 1276 of SEQ ID NO: 1 or the amino acid sequence of SEQ ID NO: 1.

In some embodiments, the nucleic acid molecule encoding the SARS-CoV-2 antigen is incorporated into a viral particle.

Further provided are vectors comprising the nucleic acid molecule encoding the SARS-CoV-2 antigen. In some embodiments, the vector is an expression vector. The nucleic acid molecule may be operably linked to a regulatory element selected from a promoter and a poly-adenylation signal. The expression vector may be a plasmid or viral vector. An exemplary vector is pGX9541.

Immunogenic compositions comprising an effective amount of the vector or viral particle are disclosed. The immunogenic composition may comprise a pharmaceutically acceptable excipient, such as but not limited to, a buffer. The buffer may optionally be saline-sodium citrate buffer. In some embodiments, the immunogenic compositions comprise an adjuvant. An exemplary immunogenic composition is the INO-4803 drug product (or INO-4803 vaccine).

Also provided herein are SARS-CoV-2 spike antigens. According to some embodiments, the SARS-CoV-2 spike antigen is a consensus antigen. In some embodiments, the SARS-CoV-2 spike antigen comprises: the amino acid sequence set forth in residues 19 to 1276 of SEQ ID NO: 1 or the amino acid sequence of SEQ ID NO: 1.

Further provided herein are vaccines for the prevention or treatment of Severe Acute Respiratory Syndrome coronavirus 2 (SARS-CoV-2) infection. The SARS-CoV-2 infection prevented or treated in accordance with the invention includes the original Wuhan strain (WT), as well as variant strains including but not limited to variants of concern such as the B.1.1.7 (United Kingdom; Alpha) variant, the B.1.351 (South African; Beta) variant, the P.1 (Brazilian; Gamma) variant, the B.1.617.2 (Delta) variant, and the B.1.1.529 (Omicron) variant. The vaccines comprise an effective amount of any one or combination of the aforementioned nucleic acid molecules, vectors, or antigens. According to some embodiments, the vaccine further comprises a pharmaceutically acceptable excipient and/or adjuvant. The pharmaceutically acceptable excipient may be a buffer, optionally saline-sodium citrate buffer. According to some embodiments, the vaccine further comprises an adjuvant.

Methods of inducing an immune response against Severe Acute Respiratory Syndrome coronavirus 2 (SARS-CoV-2) in a subject in need thereof are further provided. In some embodiments, the methods of inducing an immune response comprise administering an effective amount of any one or combination of the aforementioned nucleic acid molecules, vectors, immunogenic compositions, antigens, or vaccines to the subject. Also provided herein are methods of protecting a subject in need thereof from infection with SARS-CoV-2, the method comprising administering an effective amount of any one or combination of the aforementioned nucleic acid molecules, vectors, immunogenic compositions, antigens, or vaccines to the subject. Further provided are methods of treating SARS-CoV-2 infection in a subject in need thereof, the method comprising administering an effective amount of any one or combination of the aforementioned nucleic acid molecules, vectors, immunogenic compositions, antigens, or vaccines to the subject. The SARS-CoV-2 infection prevented or treated in accordance with the invention includes the original Wuhan strain (WT), as well as variant strains including but not limited to variants of concern such as the B.1.1.7 (United Kingdom; Alpha) variant, the B.1.351 (South African; Beta) variant, the P.1 (Brazilian; Gamma) variant, the B.1.617.2 (Delta) variant, and the B.1.1.529 (Omicron) variant. Also provided herein are methods for treating or protecting a subject in need thereof against a disease or disorder associated with SARS-CoV-2 infection, the method comprising administering an effective amount of any one or combination of the aforementioned nucleic acid molecules, vectors, immunogenic compositions, antigens, or vaccines to the subject. In some embodiments, the disease or disorder associated with SARS-CoV-2 infection is Coronavirus Disease 2019 (COVID-19), Multisystem inflammatory syndrome in adults (MIS-A), or Multisystem inflammatory syndrome in children (MIS-C). In any of these methods, the administering may include at least one of electroporation and injection. According to some embodiments, the administering comprises parenteral administration, for example by intradermal, intramuscular, or subcutaneous injection, optionally followed by electroporation. In some embodiments of the disclosed methods, an initial dose of about 0.5 mg to about 2.0 mg of the nucleic acid molecule is administered to the subject, optionally the initial dose is 0.5 mg, 1.0 mg or 2.0 mg of the nucleic acid molecule. The methods may further involve administration of a subsequent dose of about 0.5 mg to about 2.0 mg of the nucleic acid molecule to the subject about four weeks after the initial dose, optionally wherein the subsequent dose is 0.5 mg,1.0 mg or 2.0 mg of the nucleic acid molecule. In still further embodiments, the methods involve administration of one or more further subsequent doses of about 0.5 mg to about 2.0 mg of the nucleic acid molecule to the subject at least twelve weeks after the initial dose, optionally wherein the further subsequent dose is 0.5 mg, 1.0 mg, or 2.0 mg of the nucleic acid molecule. According to some embodiments, the nucleic acid molecule comprises: the nucleic acid sequence of nucleotides 55 to 3828 of SEQ ID NO: 2; the nucleic acid sequence of SEQ ID NO: 2; the nucleic acid sequence of SEQ ID NO: 3; or a nucleic acid molecule encoding a SARS-CoV-2 spike antigen, wherein the SARS-CoV-2 spike antigen comprises the amino acid sequence set forth in residues 19 to 1276 of SEQ ID NO: 1 or the amino acid sequence of SEQ ID NO: 1. For example, pGX9541, INO-4803 drug product, or a biosimilar thereof may be administered in accordance with any of the aforementioned methods.

Also provided herein are uses of any one or combination of the disclosed nucleic acid molecules, vectors, immunogenic compositions, antigens, or vaccines in a method of inducing an immune response against Severe Acute Respiratory Syndrome coronavirus 2 (SARS-CoV-2) in a subject in need thereof. Further provided are uses of any one or combination of the disclosed nucleic acid molecules, vectors, immunogenic compositions, antigens, or vaccines in a method of protecting a subject from infection with Severe Acute Respiratory Syndrome coronavirus 2 (SARS-CoV-2). Also provided herein are uses of any one or combination of the disclosed nucleic acid molecules, vectors, immunogenic compositions, antigens, or vaccines in a method of treating a subject in need thereof against SARS-CoV-2 infection. The SARS-CoV-2 infection prevented or treated in accordance with the invention includes the original Wuhan strain (WT), as well as variant strains including but not limited to variants of concern such as the B.1.1.7 (United Kingdom; Alpha) variant, the B.1.351 (South African; Beta) variant, the P.1 (Brazilian; Gamma) variant, the B.1.617.2 (Delta) variant, and the B.1.1.529 (Omicron) variant. Also provided herein are uses of any one or combination of the disclosed nucleic acid molecules, vectors, immunogenic compositions, antigens, or vaccines in a method of treating or protecting a subject in need thereof against a disease or disorder associated with SARS-CoV-2 infection. In some embodiments, the disease or disorder associated with SARS-CoV-2 infection is Coronavirus Disease 2019 (COVID-19), Multisystem inflammatory syndrome in adults (MIS-A), or Multisystem inflammatory syndrome in children (MIS-C). In accordance with any of these uses, the nucleic acid molecule, the vector, the immunogenic composition, the antigen, or the vaccine may be administered to the subject by at least one of electroporation and injection. In some embodiments, the nucleic acid molecule, the vector, the immunogenic composition, the antigen, or the vaccine is administered parenterally to the subject followed by electroporation. In some embodiments of the disclosed uses, an initial dose of about 0.5 mg to about 2.0 mg of the nucleic acid molecule is administered to the subject, optionally the initial dose is 0.5 mg, 1.0 mg or 2.0 mg of the nucleic acid molecule. The uses may further involve administration of a subsequent dose of about 0.5 mg to about 2.0 mg of the nucleic acid molecule to the subject about four weeks after the initial dose, optionally wherein the subsequent dose is 0.5 mg,1.0 mg or 2.0 mg of the nucleic acid molecule. In still further embodiments, the uses involve administration of one or more further subsequent doses of about 0.5 mg to about 2.0 mg of the nucleic acid molecule to the subject at least twelve weeks after the initial dose, optionally wherein the further subsequent dose is 0.5 mg, 1.0 mg, or 2.0 mg of the nucleic acid molecule. According to some embodiments, the nucleic acid molecule administered in accordance with any of the aforementioned uses comprises: the nucleic acid sequence of nucleotides 55 to 3828 of SEQ ID NO: 2; the nucleic acid sequence of SEQ ID NO: 2; the nucleic acid sequence of SEQ ID NO: 3; or a nucleic acid molecule encoding a SARS-CoV-2 spike antigen, wherein the SARS-CoV-2 spike antigen comprises the amino acid sequence set forth in residues 19 to 1276 of SEQ ID NO: 1 or the amino acid sequence of SEQ ID NO: 1. For example, pGX9541, INO-4803 drug product or a biosimilar thereof may be administered in accordance with any of the aforementioned uses.

Further provided herein are uses of any one or combination of the disclosed nucleic acid molecules, vectors, immunogenic compositions, antigens, or vaccines in the preparation of a medicament. In some embodiments, the medicament is for treating or protecting against infection with Severe Acute Respiratory Syndrome coronavirus 2 (SARS-CoV-2). The SARS-CoV-2 infection prevented or treated in accordance with the invention includes the original Wuhan strain (WT), as well as variant strains including but not limited to variants of concern such as the B.1.1.7 (United Kingdom; Alpha) variant, the B.1.351 (South African; Beta) variant, the P.1 (Brazilian; Gamma) variant, the B.1.617.2 (Delta) variant, and the B.1.1.529 (Omicron) variant. In some embodiments, the medicament is for treating or protecting against a disease or disorder associated with SARS-CoV-2 infection. In some embodiments, the medicament is for treating or protecting against Coronavirus Disease 2019 (COVID-19), Multisystem inflammatory syndrome in adults (MIS-A), or Multisystem inflammatory syndrome in children (MIS-C). According to some embodiments, the nucleic acid molecule administered in accordance with any of the aforementioned uses comprises: the nucleic acid sequence of nucleotides 55 to 3828 of SEQ ID NO: 2; the nucleic acid sequence of SEQ ID NO: 2; the nucleic acid sequence of SEQ ID NO: 3; or a nucleic acid molecule encoding a SARS-CoV-2 spike antigen, wherein the SARS-CoV-2 spike antigen comprises the amino acid sequence set forth in residues 19 to 1276 of SEQ ID NO: 1 or the amino acid sequence of SEQ ID NO: 1. For example, pGX9541, INO-4803 drug product or a biosimilar thereof may be administered in accordance with any of the aforementioned uses.

### BRIEF DESCRIPTION OF THE DRAWINGS

FIG. 1 provides a diagrammatic representation of DNA Plasmid pGX9541.

FIG. 2 provides a diagrammatic representation of the modified pVAX1 backbone (pGX0001).

FIG. 3 illustrates construction of plasmid pGX9541.

FIG. 4 illustrates the design of the study to evaluate the immunogenicity of INO-4803 (pGX9541 plasmid) in female BALB/c mice. On day 0 mice received a single dose of DNA plasmid delivered by intramuscular injection followed by electroporation with CELLECTRA® 3P. On day 19, mice were euthanized, and IFNγ ELISpot, antibody binding and pseudovirus neutralization assays were performed on the collected samples.

FIGS. 5A-5C show humoral and cellular immune responses measured after single administration of INO-4803 in BALB/c mice. On day 19, splenocytes and sera from immunized mice were collected to assess cellular and humoral responses to SARS-CoV-2 spike variants. FIG. 5A shows SARS-CoV-2 Spike specific T cell responses as measured by IFNγ ELISpot assay for individual mice. T cell responses were measured by IFN-γ ELISpot in splenocytes stimulated with WT or Omicron spike peptides. Bars represent mean ± standard deviation (SD). FIG. 5B shows full-length Spike Protein antigen binding of IgG in serum samples from mice at 19 days post primary immunization. Data shown represent geometric mean titer values (GMT+/- 95% confidence interval [CI]) for each group of 6 mice administered pGX0001, pGX9501 (INO-4800), or pGX9541. FIG. 5C shows WT and Omicron SARS-CoV-2 pseudovirus neutralization ID₅₀ titers in mouse sera. Each data point represents the mean of technical duplicates for each animal (n=6/group) and the geometric mean ± 95% CI is presented. Dashed lines represent the limit of detection (LOD) of the assay.

FIG. 6 illustrates the study design to evaluate immunogenicity of INO-4803 (pGX9541) in C57BL/6 mice. On day 0, mice received a single dose of DNA plasmid delivered by intramuscular injection followed by electroporation with CELLECTRA® 3P. On day 19, mice were euthanized, and IFNγ ELISpot, antibody binding and pseudovirus neutralization assays were performed on the collected samples.

FIGS. 7A-7C show humoral and cellular immune responses measured after single administration of INO-4803 in C57BL/6 mice. On day 19, splenocytes and sera from immunized mice were collected to assess cellular and humoral responses to SARS-CoV-2 spike plasmids. FIG. 7A shows SARS-CoV-2 Spike specific T cell responses as measured by IFNγ ELISpot assay for individual mice. T cell responses were measured by IFN-γ ELISpot in splenocytes stimulated with WT, Omicron or Delta spike peptides. Bars represent mean ± SD. FIG. 7B shows Full-length Spike or receptor binding domain (RBD) antigen binding of IgG in serum samples from mice at 19 days post primary immunization. Data shown represent geometric mean titer values (GMT+/- 95% CI) for each group of 6 mice administered pGX0001, pGX9501, and pGX9541. FIG. 7C shows WT, Omicron, and Delta SARS-CoV-2 pseudovirus neutralization ID₅₀ titers in mouse sera. Each data point represents the mean of technical duplicates for each animal (n=6/group) and the geometric mean ± 95% CI is presented. Dashed lines represent the limit of detection (LOD) of the assay.

FIG. 8 provides the study design to evaluate immunogenicity of INO-4803 (pGX9541) in Syrian Golden hamsters. On days 0 and 21, animals received DNA plasmid delivered by intradermal injection followed by electroporation with CELLECTRA® 3P. On day 35, animals were euthanized, and IFNγ ELISpot, and pseudovirus neutralization assays were performed on the collected samples.

FIGS. 9A and 9B show humoral and cellular immune responses measured in Syrian Golden Hamsters immunized with INO-4803. FIG. 9A shows SARS-CoV-2 Spike specific T cell responses as measured by IFNγ ELISpot assay for individual hamsters. T cell responses were measured by IFN-γ ELISpot in splenocytes stimulated with WT or Omicron spike peptides. Bars represent mean ± SD. FIG. 9B shows WT and Omicron SARS-CoV-2 pseudovirus neutralization ID₅₀ titers in hamster sera collected after one (day 21) or two (day 35) immunizations. Each data point represents the mean of technical duplicates for each animal (n=6/group) and the geometric mean ± 95% CI is presented. Dashed lines represent the limit of detection (LOD) of the assay.

### DETAILED DESCRIPTION OF ILLUSTRATIVE EMBODIMENTS

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

When a range of numerical values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

### Certain Terminology

As used herein, the term “about” when used in reference to numerical ranges, cutoffs, or specific values is used to indicate that the recited values may vary by up to as much as 10% from the listed value. Thus, the term “about” is used to encompass variations of ± 10% or less, variations of ± 5% or less, variations of ± 1% or less, variations of ± 0.5% or less, or variations of ± 0.1% or less from the specified value.

As used herein, the term “at least one” means “one or more.”

The term “comprising” is intended to include examples encompassed by the terms “consisting essentially of” and “consisting of”; similarly, the term “consisting essentially of” is intended to include examples encompassed by the term “consisting of.” The present disclosure also contemplates other embodiments “comprising,” “consisting of” and “consisting essentially of,” the embodiments or elements presented herein, whether explicitly set forth or not.

“Adjuvant” as used herein means any molecule added to an immunogenic composition or vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′) 2, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

The term “biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extent the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may further include sequences that encode signal peptides, e.g., an IgE leader sequence.

“Complement” or “complementary” as used herein means Watson-Crick (e.g., A-T/U and C-G) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below.

“Fragment” or “immunogenic fragment” with respect to polypeptide sequences means a polypeptide capable of eliciting an immune response in a mammal that cross reacts with a reference full-length SARS-CoV-2 antigen. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host’s immune system, e.g., that of a mammal, in response to the introduction of antigen. The immune response can be in the form of a cellular or humoral response, or both.

The INO-4800 drug product contains 10 mg/mL of the DNA plasmid pGX9501 (or INO-4800) in 1X SSC buffer (150 mM sodium chloride and 15 mM sodium citrate).

The INO-4802 drug product contains 10 mg/mL of the DNA plasmid pGX9527 (or INO-4802) in 1X SSC buffer (150 mM sodium chloride and 15 mM sodium citrate).

The INO-4803 drug product contains 10 mg/mL of the DNA plasmid pGX9541 (or INO-4803) in 1X SSC buffer (150 mM sodium chloride and 15 mM sodium citrate).

“Nucleic acid” or “oligonucleotide” or “polynucleotide” or “nucleic acid molecule” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions. Nucleic acids can be single stranded or double-stranded or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, and CMV IE promoter.

“Signal peptide” and “leader sequence” are used interchangeably herein and refer to an amino acid sequence that can be linked at the amino terminus of a SARS-CoV-2 protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants or is in need of being immunized with a herein described immunogenic composition or vaccine. The mammal can be a human, chimpanzee, guinea pig, dog, cat, horse, cow, mouse, hamster, rabbit, or rat. Thus, the methods are applicable to human and nonhuman animals, although preferably used most preferably with humans. “Subject” and “patient” are used interchangeably herein.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of an animal from a disease through means of preventing, suppressing, repressing, or completely eliminating the disease. Preventing the disease involves administering an immunogenic composition or a vaccine of the present invention to an animal prior to onset of the disease. Suppressing the disease involves administering an immunogenic composition or a vaccine of the present invention to an animal after induction of the disease but before its clinical appearance. Repressing the disease involves administering an immunogenic composition or a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof.

“Variant” used herein with respect to a peptide or polypeptide that refers to a peptide or polypeptide that differs in amino acid sequence by the insertion, deletion, or conservative substitution of amino acids, but retains at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Nucleic Acid Molecules, Antigens, and Immunogenic Compositions

Provided herein are immunogenic compositions, such as vaccines, comprising a nucleic acid molecule encoding a SARS-CoV-2 spike antigen, a fragment thereof, a variant thereof, or a combination thereof. Also provided herein are immunogenic compositions, such as vaccines, comprising a SARS-CoV-2 spike antigen, a fragment thereof, a variant thereof, or a combination thereof. The immunogenic compositions can be used to protect against and treat any number of strains of SARS-CoV-2, thereby treating, preventing, and/or protecting against SARS-CoV-2-based pathologies. The immunogenic compositions can significantly induce an immune response of a subject administered the immunogenic compositions, thereby protecting against and/or treating SARS-CoV-2 infection.

The immunogenic composition can be a DNA vaccine, a peptide vaccine, or a combination DNA and peptide vaccine. The DNA vaccine can include a nucleic acid molecule encoding the SARS-CoV-2 spike antigen. The nucleic acid molecule can be DNA, RNA, cDNA, a variant thereof, a fragment thereof, or a combination thereof. The nucleic acid molecule can also include additional sequences that encode linker, leader, or tag sequences that are linked to the nucleic acid molecule encoding the SARS-CoV-2 spike antigen by a peptide bond. The peptide vaccine can include a SARS-CoV-2 antigenic peptide, a SARS-CoV-2 antigenic protein, a variant thereof, a fragment thereof, or a combination thereof. The combination DNA and peptide vaccine can include the above-described nucleic acid molecule encoding the SARS-CoV-2 spike antigen and the SARS-CoV-2 spike antigenic peptide or protein, in which the SARS-CoV-2 spike antigenic peptide or protein and the encoded SARS-CoV-2 spike antigen have the same or different amino acid sequence.

The disclosed immunogenic compositions can elicit both humoral and cellular immune responses that target the SARS-CoV-2 spike antigen in the subject administered the immunogenic composition. The disclosed immunogenic compositions can elicit neutralizing antibodies and immunoglobulin G (IgG) antibodies that are reactive with the SARS-CoV-2 spike antigen. The immunogenic composition can also elicit CD8+ and CD4+ T cell responses that are reactive to the SARS-CoV-2 spike antigen and produce interferon-gamma (IFN-γ), interleukin-2 (IL-2), TNFα, interleukin-4 (IL-4), circulating T follicular helper (Tfh) cells, or any combination thereof.

The immunogenic composition can induce a humoral immune response in the subject administered the immunogenic composition. The induced humoral immune response can be specific for the SARS-CoV-2 spike antigen. The induced humoral immune response can be reactive with the SARS-CoV-2 spike antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the immunogenic composition can include an increased level of neutralizing antibodies associated with the subject administered the immunogenic composition as compared to a subject not administered the immunogenic composition. The neutralizing antibodies can be specific for the SARS-CoV-2 spike antigen. The neutralizing antibodies can be reactive with the SARS-CoV-2 spike antigen. The neutralizing antibodies can provide protection against and/or treatment of SARS-CoV-2 infection and its associated pathologies in the subject administered the immunogenic composition.

The humoral immune response induced by the immunogenic composition can include an increased level of IgG antibodies associated with the subject administered the immunogenic composition as compared to a subject not administered the immunogenic composition and/or as compared to a subject administered pGX9501 (INO-4800). These IgG antibodies can be specific for the SARS-CoV-2 spike antigen. These IgG antibodies can be reactive with the SARS-CoV-2 spike antigen. The level of IgG antibody associated with the subject administered the immunogenic composition can be increased by about 1.5-fold to about 16-fold, about 2-fold to about 12-fold, or about 3-fold to about 10-fold as compared to the subject not administered the immunogenic composition and/or as compared to a subject administered pGX9501 (INO-4800). The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the immunogenic composition and/or as compared to a subject administered pGX9501 (INO-4800).

The immunogenic composition can induce a cellular immune response in the subject administered the immunogenic composition. The induced cellular immune response can be specific for the SARS-CoV-2 spike antigen. The induced cellular immune response can be reactive to the SARS-CoV-2 spike antigen. The induced cellular immune response can include eliciting a CD8+ T cell response. The elicited CD8+ T cell response can be reactive with the SARS-CoV-2 spike antigen. The elicited CD8+ T cell response can be polyfunctional. The induced cellular immune response can include eliciting a CD8+ T cell response, in which the CD8+ T cells produce interferon-gamma (IFN-γ), interleukin-2, and/or upregulation of CD107a.

The induced cellular immune response can include an increased CD8+ T cell response associated with the subject administered the immunogenic composition as compared to the subject not administered the immunogenic composition and/or as compared to a subject administered pGX9501 (INO-4800). The CD8+ T cell response associated with the subject administered the immunogenic composition can be increased by about 2-fold to about 30-fold, about 3-fold to about 25-fold, or about 4-fold to about 20-fold as compared to the subject not administered the immunogenic composition and/or as compared to a subject administered pGX9501 (INO-4800). The CD8+ T cell response associated with the subject administered the immunogenic composition can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the immunogenic composition and/or as compared to a subject administered pGX9501 (INO-4800).

The cellular immune response induced by the immunogenic composition can include eliciting a CD4+ T cell response. The elicited CD4+ T cell response can be reactive with the SARS-CoV-2 antigen. The elicited CD4+ T cell response can be polyfunctional. The induced cellular immune response can include eliciting a CD4+ T cell response, in which the CD4+ T cells produce IFN-y, interleukin-2 (IL-2), interleukin-4 (IL-4), Tumour Necrosis Factor alpha (TNFα), or any combination thereof.

The cellular immune response induced by the immunogenic composition can include an increase in circulating Tfh (CXCR5+ PD-1+) cells.

The immunogenic composition of the present invention can have features required of effective immunogenic compositions such as being safe so the immunogenic composition itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The immunogenic composition can further induce an immune response when administered to different tissues such as the muscle or skin. The immunogenic composition can further induce an immune response when parenterally administered, for example by subcutaneous, intradermal, or intramuscular injection, optionally followed by electroporation as described herein.

### A. SARS-CoV-2 Antigen and Nucleic Acid Molecules Encoding the Same

As described above, provided herein are immunogenic compositions comprising a nucleic acid molecule encoding a SARS-CoV-2 spike antigen, a fragment thereof, a variant thereof, or a combination thereof. Also provided herein are immunogenic compositions comprising a SARS-CoV-2 spike antigen, a fragment thereof, a variant thereof, or a combination thereof.

The SARS-CoV-2 spike antigen is capable of eliciting an immune response in a mammal against one or more SARS-CoV-2 strains. According to some embodiments, the SARS-CoV-2 strain or variant is SARS-CoV-2 Omicron variant (B.1.1.529). The SARS-CoV-2 spike antigen can comprise an epitope(s) that makes it particularly effective as an immunogen against which an anti- SARS-CoV-2 immune response can be induced.

The SARS-CoV-2 antigen can be a consensus antigen derived from one or more strains of SARS-CoV-2. In some embodiments, the SARS-CoV-2 antigen is a SARS-CoV-2 consensus spike antigen. The SARS-CoV-2 consensus spike antigen can be derived from the sequences of spike antigens from one or more strains of SARS-CoV-2, and thus, the SARS-CoV-2 consensus spike antigen is unique. According to some embodiments, the immunogenic compositions of the present invention are widely applicable to multiple strains of SARS-CoV-2 because of the unique sequences of the SARS-CoV-2 consensus spike antigen. These unique sequences allow the vaccine to be protective against multiple strains of SARS-CoV-2, including genetically diverse variants of SARS-CoV-2.

Nucleic acid molecules encoding the SARS-CoV-2 antigen can be modified for improved expression. Modification can include codon optimization, RNA optimization, addition of a kozak sequence for increased translation initiation, and/or the addition of an immunoglobulin leader sequence to increase the immunogenicity of the SARS-CoV-2 spike antigen. The SARS-CoV-2 spike antigen can comprise a signal peptide such as an immunoglobulin signal peptide, for example, but not limited to, an immunoglobulin E (IgE) or immunoglobulin (IgG) signal peptide. In some embodiments, the SARS-CoV-2 spike antigen can comprise a hemagglutinin (HA) tag. The SARS-CoV-2 spike antigen can be designed to elicit stronger and broader cellular and/or humoral immune responses than a corresponding codon optimized spike antigen.

In some embodiments, the SARS-CoV-2 spike antigen comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of residues 19 to 1276 of SEQ ID NO: 1. In some embodiments the SARS-CoV-2 spike antigen comprises the amino acid sequence set forth in residues 19 to 1276 of SEQ ID NO: 1. In some embodiments, the SARS-CoV-2 spike antigen comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of SEQ ID NO: 1. In some embodiments the SARS-CoV-2 spike antigen comprises the amino acid sequence of SEQ ID NO: 1.

In some embodiments the nucleic acid molecule encoding the SARS-CoV-2 spike antigen comprises the nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity to the sequence set forth in nucleotides 55 to 3828 of SEQ ID NO: 2, SEQ ID NO: 2, or SEQ ID NO: 3.

In some embodiments the SARS-CoV-2 spike antigen is operably linked to an IgE leader sequence. In some such embodiments, the SARS-CoV-2 spike antigen comprises the amino acid sequence set forth in SEQ ID NO: 1. In some embodiments, the SARS-CoV-2 spike antigen having an IgE leader sequence is encoded by the nucleotide sequence set forth in SEQ ID NO:2 or SEQ ID NO: 3.

Immunogenic fragments of SEQ ID NO:1 are provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:1. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:1 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:1. Some embodiments relate to immunogenic fragments that have 96% homology to the immunogenic fragments of consensus protein sequences herein. Some embodiments relate to immunogenic fragments that have 97% homology to the immunogenic fragments of consensus protein sequences herein. Some embodiments relate to immunogenic fragments that have 98% homology to the immunogenic fragments of consensus protein sequences herein. Some embodiments relate to immunogenic fragments that have 99% homology to the immunogenic fragments of consensus protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:1. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:1. Immunogenic fragments can be at least 95%, at least 96%, at least 97% at least 98% or at least 99% homologous to fragments of SEQ ID NO:1. In some embodiments, immunogenic fragments include sequences that encode a leader sequence, such as for example an immunoglobulin leader, such as the IgE leader. In some embodiments, fragments are free of coding sequences that encode a leader sequence.

### B. Vector

The immunogenic compositions can comprise one or more vectors that include a nucleic acid molecule encoding the SARS-CoV-2 spike antigen. The one or more vectors can be capable of expressing the spike antigen. The vector can have a nucleic acid sequence containing an origin of replication. The vector can be a plasmid, bacteriophage, bacterial artificial chromosome or yeast artificial chromosome. The vector can be either a self-replicating extrachromosomal vector or a vector which integrates into a host genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

### Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

### Circular and Linear Vectors

The vector may be a circular plasmid, which may transform a target cell by integration into the cellular genome or exist extrachromosomally (e.g., autonomous replicating plasmid with an origin of replication).

The vector can be pVAX, pcDNA3.0, pGX0001, or provax, or any other expression vector capable of expressing DNA encoding the antigen and enabling a cell to translate the sequence to an antigen that is recognized by the immune system.

Also provided herein is a linear nucleic acid immunogenic composition, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

The LEC may be derived from any plasmid capable of being linearized. The plasmid may be capable of expressing the antigen. The plasmid can be pNP (Puerto Rico/34) or pM2 (New Caledonia/99). The plasmid may be WLV009, pVAX, pGX0001, pcDNA3.0, or provax, or any other expression vector capable of expressing DNA encoding the antigen and enabling a cell to translate the sequence to an antigen that is recognized by the immune system.

The LEC can be perM2. The LEC can be perNP. perNP and perMR can be derived from pNP (Puerto Rico/34) and pM2 (New Caledonia/99), respectively.

### Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

### C. Excipients and Other Components of the Immunogenic Compositions

The immunogenic compositions may further comprise a pharmaceutically acceptable excipient. The pharmaceutically acceptable excipient can be functional molecules such as vehicles, carriers, buffers, or diluents. As used herein. “buffer” refers to a buffered solution that resists changes in pH by the action of its acid-base conjugate components. The buffer generally has a pH from about 4.0 to about 8.0, for example from about 5.0 to about 7.0. In some embodiments, the buffer is saline-sodium citrate (SSC) buffer. In some embodiments in which the immunogenic composition comprises a nucleic acid molecule encoding a SARS-CoV-2 spike antigen as described above, the immunogenic composition comprises 10 mg/ml of vector in buffer, for example but not limited to SSC buffer. In some embodiments, the immunogenic composition comprises 10 mg/mL of the DNA plasmid pGX9541 in buffer. In some embodiments, the immunogenic composition is stored at about 2° C. to about 8° C. In some embodiments, the immunogenic composition is stored at room temperature. The immunogenic composition may be stored for at least a year at room temperature. In some embodiments, the immunogenic composition is stable at room temperature for at least a year, wherein stability is defined as a supercoiled plasmid percentage of at least about 80%. In some embodiments, the supercoiled plasmid percentage is at least about 85% following storage for at least a year at room temperature.

The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent may be a polyanion, polycation, including poly-L-glutamate (LGS), or lipid. The transfection facilitating agent is poly-L-glutamate, and the poly-L-glutamate may be present in the immunogenic composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid immunogenic compositions may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the immunogenic composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in an alternative plasmid or are delivered as proteins in combination with the plasmid above in the immunogenic composition. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), interleukin (IL)-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The immunogenic composition can be formulated according to the mode of administration to be used. According to some embodiments, the immunogenic composition is formulated in a buffer, optionally saline-sodium citrate buffer. For example, the immunogenic composition may formulated at a concentration of 10 mg nucleic acid molecule per milliliter of buffer, optionally a sodium salt citrate buffer. An injectable immunogenic pharmaceutical composition can be sterile, pyrogen free and particulate free. An isotonic formulation or solution can be used. Additives for isotonicity can include sodium chloride, dextrose, mannitol, sorbitol, and lactose. The immunogenic composition can comprise a vasoconstriction agent. The isotonic solutions can include phosphate buffered saline. Immunogenic compositions can further comprise stabilizers including gelatin and albumin. The stabilizers can allow the formulation to be stable at room or ambient temperature for extended periods of time, including LGS or polycations or polyanions.

### Methods of Vaccination

Also provided herein are methods of treating, protecting against, and/or preventing disease in a subject in need thereof by administering the immunogenic composition to the subject. Administration of the immunogenic composition to the subject can induce or elicit an immune response in the subject. The induced immune response can be used to treat, prevent, and/or protect against disease, for example, pathologies relating to SARS-CoV-2 infection. The induced immune response in the subject administered the immunogenic composition can provide resistance to one or more SARS-CoV-2 strains, for example, SARS-CoV-2 Omicron (B.1.1.529) variant.

The induced immune response can include an induced humoral immune response and/or an induced cellular immune response. The humoral immune response can be induced by about 1.5-fold to about 16-fold, about 2-fold to about 12-fold, or about 3-fold to about 10-fold relative to the subject’s baseline, to a subject who is not administered the immunogenic composition, or to a subject who has been administered INO-4800 (pGX9501). The induced humoral immune response can include IgG antibodies and/or neutralizing antibodies that are reactive to the antigen. The induced cellular immune response can include a CD8+ T cell response, which is induced by about 2-fold to about 30-fold, about 3-fold to about 25-fold, or about 4-fold to about 20-fold. The induced cellular immune response can include a CD4+ T cell response, which is induced by about 2-fold to about 30-fold, about 3-fold to about 25-fold, or about 4-fold to about 20-fold. The induced cellular immune response can include an increase in Tfh cells by about 2-fold to about 30-fold, about 3-fold to about 25-fold, or about 4-fold to about 20-fold.

The vaccine dose can be between 1 µg to 10 mg active component/kg body weight/time, and can be 20 µg to 10 mg component/kg body weight/time. The vaccine can be administered every 21, 22, 23, 24, 25, 26, 27, 28, 29, 30, 31, 32, 33, 34, 35, 36, 37, 38, 39, 40 or more days or every 3, 4, 5, 6, 7, 8, 9, 10, 11, 12, 13, 14, 15, 16, 17, 18, 19, 20 or more weeks. The number of vaccine doses for effective treatment can be 1, 2, 3, 4, 5, 6, 7, 8, 9, 10, or more.

### A. Administration

The immunogenic composition can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The vaccine may be administered, for example, in one, two, three, four, or more injections. In some embodiments, an initial dose of about 0.5 mg to about 2.0 mg of the nucleic acid molecule is administered to the subject. The initial dose may be administered in one, two, three, or more injections. The initial dose may be followed by administration of one, two, three, four, or more subsequent doses of about 0.5 mg to about 2.0 mg of the nucleic acid molecule about one, two, three, four, five, six, seven, eight, ten, twelve or more weeks after the immediately prior dose. Each subsequent dose may be administered in one, two, three, or more injections. In some embodiments, the immunogenic composition is administered to the subject before, with, or after an additional agent. In some embodiments, the immunogenic composition is administered as a booster following administration of an agent for the treatment of SARS-CoV-2 infection or the treatment or prevention of a disease or disorder associated with SARS-CoV-2 infection. The agent for the treatment of SARS-CoV-2 infection or the treatment or prevention of a disease or disorder associated with SARS-CoV-2 infection may be, for example but not limited to, a SARS-CoV-2 wild-type matched vaccine, pGX9501, INO-4800 drug product or a biosimilar thereof, pGX9527, or INO-4802 drug product or a biosimilar thereof. In one embodiment, the disease or disorder associated with SARS-CoV-2 infection includes, but is not limited to, to Coronavirus Disease 2019 (COVID-19). In some embodiments, the disease or disorder associated with SARS-CoV-2 infection is Multisystem inflammatory syndrome in adults (MIS-A) or Multisystem inflammatory syndrome in children (MIS-C).

The subject can be a mammal, such as a human, a horse, a nonhuman primate, a cow, a pig, a sheep, a cat, a dog, a guinea pig, a rabbit, a rat, a mouse, or a hamster.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety). Parenteral administration may optionally be followed with electroporation as described herein.

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Pat. Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject’s skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA® (Inovio Pharmaceuticals, Blue Bell Pa.) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra® device and system is described in U.S. Pat. No. 7,245,963, the contents of which are herein incorporated by reference. The CELLECTRA® device may be the CELLECTRA 2000® device or CELLECTRA® 3PSP device. The CELLECTRA® 2000 device is configured by the manufacturer to support either ID (intradermal) or IM (intramuscular) administration. The CELLECTRA® 2000 includes the CELLECTRA® Pulse Generator, the appropriate applicator, disposable sterile array and disposable sheath (ID only). The DNA plasmid is delivered separately via needle and syringe injection in the area delineated by the electrodes immediately prior to the electroporation treatment.

The MID may be an Elgen 1000 system (Inovio Pharmaceuticals). The Elgen 1000 system may comprise a device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or nonlinear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient’s skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

### Use in Combination

In some embodiments, the present invention provides a method of treating, protecting against, and/or preventing a SARS-CoV-2 infection, or treating, protecting against, and/or preventing a disease or disorder associated with SARS-CoV-2 infection, in a subject in need thereof by administering a combination of a nucleic acid molecule encoding a SARS-CoV-2 spike antigen as disclosed herein, or fragment or variant thereof, in combination with one or more additional agents for treating, protecting against, and/or preventing of SARS-CoV-2 infection or the treatment or prevention of disease or disorder associated with SARS-CoV-2 infection. According to some embodiments, the SARS-CoV-2 is SARS-CoV-2 Omicron (B.1.1.529) variant. In some embodiments, the disease or disorder associated with SARS-CoV-2 infection is Coronavirus Disease 2019 (COVID-19), Multisystem inflammatory syndrome in adults (MIS-A), or Multisystem inflammatory syndrome in children (MIS-C).

The nucleic acid molecule encoding a SARS-CoV-2 spike antigen and additional agent may be administered using any suitable method such that a combination of the nucleic acid molecule encoding a SARS-CoV-2 spike antigen and the additional agent are both present in the subject. In one embodiment, the method may comprise administration of a first composition comprising an agent for the prevention or treatment of SARS-CoV-2 infection or the treatment or prevention of disease or disorder associated with SARS-CoV-2 infection and administration of a second composition comprising a nucleic acid molecule encoding a SARS-CoV-2 spike antigen as disclosed herein less than 1, less than 2, less than 3, less than 4, less than 5, less than 6, less than 7, less than 8, less than 9 or less than 10 days following administration of the first composition comprising the agent for the treatment or prevention of SARS-CoV-2 infection or the treatment or prevention of disease or disorder associated with SARS-CoV-2 infection. In one embodiment, the method may comprise administration of a first composition comprising a nucleic acid molecule encoding a SARS-CoV-2 spike antigen as disclosed herein and administration of a second composition comprising an agent for the treatment or prevention of SARS-CoV-2 infection or the treatment or prevention of disease or disorder associated with SARS-CoV-2 infection less than 1, less than 2, less than 3, less than 4, less than 5, less than 6, less than 7, less than 8, less than 9 or less than 10 days following administration of the nucleic acid molecule encoding a SARS-CoV-2 spike antigen. In one embodiment, the method may comprise administration of a first composition comprising an agent for the treatment or prevention of SARS-CoV-2 infection or the treatment or prevention of disease or disorder associated with SARS-CoV-2 infection and a second composition comprising a nucleic acid molecule encoding a SARS-CoV-2 spike antigen as disclosed herein concurrently. In one embodiment, the method may comprise administration of a single composition comprising an agent for the treatment or prevention of SARS-CoV-2 infection or the treatment or prevention of disease or disorder associated with SARS-CoV-2 infection and a nucleic acid molecule encoding a SARS-CoV-2 spike antigen as disclosed herein. In accordance with the disclosed methods, the nucleic acid molecule encoding a SARS-CoV-2 spike antigen comprises the nucleic acid sequence of nucleotides 55 to 3828 of SEQ ID NO: 2, the nucleic acid sequence of SEQ ID NO: 2, the nucleic acid sequence of SEQ ID NO: 3, pGX9541, INO-4803 or a biosimilar thereof.

In some embodiments, the agent for the treatment or prevention of SARS-CoV-2 infection or the treatment or prevention of disease or disorder associated with SARS-CoV-2 infection is a therapeutic agent. In one embodiment, the therapeutic agent is an antiviral agent. In one embodiment, the therapeutic agent is an antibiotic agent.

Non-limiting examples of antibiotics that can be used in combination with the a nucleic acid molecule encoding a SARS-CoV-2 antigen of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

### Administration as a Booster

In one embodiment, the immunogenic composition is administered before or as a booster vaccine following administration of an initial agent or vaccine for the treatment or prevention of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C). In accordance with the disclosed methods, the initial agent for the treatment or prevention of SARS-CoV-2 infection or the treatment or prevention of disease or disorder associated with SARS-CoV-2 infection may be, for example but not limited to, a SARS-CoV-2 wild-type matched vaccine, pGX9501, INO-4800 drug product or a biosimilar thereof, pGX9527, or INO-4802 drug product or a biosimilar thereof. In some embodiments, the booster vaccine comprises the nucleic acid sequence of nucleotides 55 to 3828 of SEQ ID NO: 2, the nucleic acid sequence of SEQ ID NO: 2, the nucleic acid sequence of SEQ ID NO: 3, pGX9541, INO-4803 or a biosimilar thereof.

In some embodiments, the booster vaccine is administered at least once, at least twice, at least 3 times, at least 4 times, or at least 5 times following administration of an initial agent or vaccine for the treatment or prevention of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C). In one embodiment, the booster vaccine is administered at least 8 hours, at least 12 hours, at least 16 hours, at least 20 hours, at least 24 hours, at least 36 hours, at least 48 hours, at least 60 hours, at least 72 hours, at least 4 days, at least 5 days, at least 6 days, at least 1 week at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 1 year or greater than 1 year following administration of an initial agent or vaccine for the treatment or prevention of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C).

### Use in Assays

In some embodiments, the nucleic acid molecules, or encoded antigens, of the invention can be used in assays in vivo or in vitro. In some embodiments, the nucleic acid molecules or encoded antigens can be used in assays for detecting the presence of anti-SARS-CoV-2 spike antibodies. Exemplary assays in which the nucleic acid molecules or encoded antigens can be incorporated into include, but are not limited to, Western blot, dot blot, surface plasmon resonance methods, Flow Cytometry methods, various immunoassays, for example, immunohistochemistry assays, immunocytochemistry assays, ELISA, capture ELISA, enzyme-linked immunospot (ELISpot) assays, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, New York; Harlow et al., 1999, Using Antibodies: A Laboratory Manual, Cold Spring Harbor Laboratory Press, NY.

In one embodiment, the SARS-CoV-2 spike antigen, or fragments thereof, of the invention can be used in an assay for intracellular cytokine staining combined with flow cytometry, to assess T-cell immune responses. This assay enables the simultaneous assessment of multiple phenotypic, differentiation and functional parameters pertaining to responding T-cells, most notably, the expression of multiple effector cytokines. These attributes make the technique particularly suitable for the assessment of T-cell immune responses induced by the vaccine of the invention.

In one embodiment, the SARS-CoV-2 spike antigen, or fragments thereof, of the invention can be used in an ELIspot assay. The ELISpot assay is a highly sensitive immunoassay that measures the frequency of cytokine-secreting cells at the single-cell level. In this assay, cells are cultured on a surface coated with a specific capture antibody in the presence or absence of stimuli. In one embodiment, the SARS-CoV-2 spike antigen, or fragments thereof, of the invention can be used as the stimulus in the ELISpot assay.

### Diagnostic Methods

In some embodiments, the invention relates to methods of diagnosing a subject as having SARS-CoV-2 infection or having SARS-CoV-2 antibodies. In some embodiments, the methods include contacting a sample from a subject with a SARS-CoV-2 spike antigen of the invention, or a cell comprising a nucleic acid molecule for expression of the SARS-CoV-2 spike antigen, and detecting binding of an anti-SARS-CoV-2 spike antibody to the SARS-CoV-2 spike antigen of the invention. In such an embodiment, binding of an anti-SARS-CoV-2 spike antibody present in the sample of the subject to the antigen, or fragment thereof, of the invention would indicate that the subject is currently infected or was previously infected with SARS-CoV-2.

### Kits and Articles of Manufacture

Provided herein is a kit, which can be used for treating a subject using the method of vaccination described above. The kit can comprise the immunogenic composition described herein.

The kit can also comprise instructions for carrying out the vaccination method described above and/or how to use the kit. Instructions included in the kit can be affixed to packaging material or can be included as a package insert. While instructions are typically written or printed materials, they are not limited to such. Any medium capable of storing instructions and communicating them to an end user is contemplated by this disclosure. Such media include, but are not limited to, electronic storage media (e.g., magnetic discs, tapes, cartridges), optical media (e.g., CD ROM), and the like. As used herein, the term “instructions” can include the address of an internet site which provides instructions.

Further provided herein are articles of manufacture containing the immunogenic composition described herein. In some embodiments, the article of manufacture is a container, such as a vial, optionally a single-use vial. In one embodiment, the article of manufacture is a single-use glass vial equipped with a stopper, which contains the immunogenic composition described herein to be administered. In some embodiments, the vial comprises a stopper, pierceable by a syringe, and a seal. In some embodiments, the article of manufacture is a syringe.

Also provided herein are articles of manufacture comprising the immunogenic composition of the invention. In some embodiments, the article of manufacture is a container holding the immunogenic composition. The container may be, for example but not limited to, a syringe or a vial. The vial may have a stopper piercable by a syringe. The immunogenic composition can be packaged in suitably sterilized containers such as ampules, bottles, or vials, either in multi-dose or in unit dosage forms. The containers are preferably hermetically sealed after being filled with a vaccine preparation. Preferably, the vaccines are packaged in a container having a label affixed thereto, which label identifies the vaccine, and bears a notice in a form prescribed by a government agency such as the United States Food and Drug Administration reflecting approval of the vaccine under appropriate laws, dosage information, and the like. The label preferably contains information about the vaccine that is useful to a health care professional administering the vaccine to a patient. The package also preferably contains printed informational materials relating to the administration of the vaccine, instructions, indications, and any necessary required warnings.

### EXAMPLES

The examples and embodiments described herein are for illustrative purposes only and various modifications or changes suggested to persons skilled in the art are to be included within the spirit and purview of this application and scope of the appended claims.

### Example 1: Design of INO-4803, SARS-CoV-2 Omicron Spike-Matched Synthetic DNA Vaccine

Synthetic DNA vaccine candidate INO-4803 (pGX9541; SEQ ID NO: 3) was designed to provide immunological coverage against the SARS-CoV-2 omicron variant of concern (VOC) which was identified circulating in South Africa in 2021 (Ren, S.Y., et al., Omicron variant (B.1.1.529) of SARS-CoV-2: Mutation, infectivity, transmission, and vaccine resistance. World J Clin Cases, 2022. 10(1): p. 1-11.). Studies revealed SARS-CoV-2 omicron VOC escaped neutralizing antibody responses elicited by the first generation of COVID-19 vaccines which had been designed to express the ancestral spike antigen (Liu, L., et al., Striking antibody evasion manifested by the Omicron variant of SARS-CoV-2. Nature, 2022. 602(7898): p. 676-681. Dejnirattisai, W., et al., Reduced neutralisation of SARS-CoV-2 omicron B.1.1.529 variant by post-immunisation serum. Lancet, 2021.).

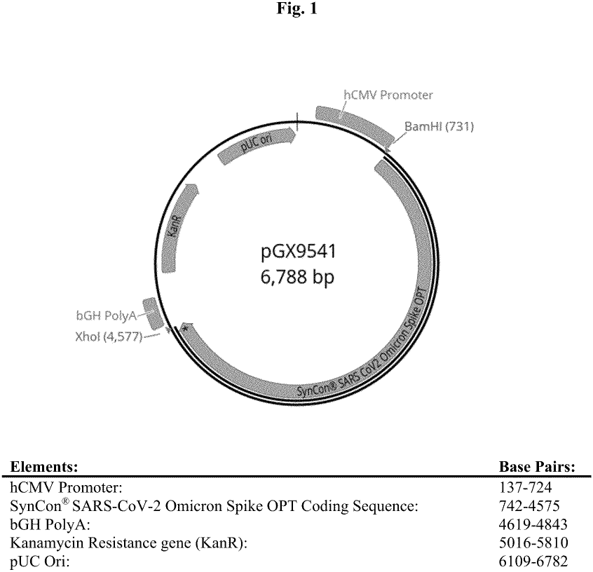

INO-4803 is a formulation of one DNA plasmid (pGX9541; SEQ ID NO: 3). pGX9541 is a DNA plasmid expressing a synthetic consensus (SynCon®) SARS-CoV-2 Spike protein (SARS-CoV-2 Omicron Spike OPT) designed to reflect the amino acid changes observed in the SARS-CoV-2 lineage B.1.1.529 (Omicron), an emerging variant worldwide, driven by a human CMV promoter (hCMV promoter), and with the bovine growth hormone 3′ end poly-adenylation signal (bGH polyA). The pGX0001 backbone includes the kanamycin resistance gene (KanR) and plasmid origin of replication (pUC ori) for production purpose. Those elements are not functional in eukaryotic cells. A schematic diagram of pGX9541 is presented in FIG. 1.

pGX9541 is derived from the cloning of the SynCon® SARS-CoV-2 Omicron Spike OPT Coding Sequence into-pGX0001 (a modified pVAX1 expression vector) between the human cytomegalovirus immediate-early promoter (hCMV promoter) and the bGH polyA. The original pVAX1 expression vector was obtained from Thermo Fisher Scientific.

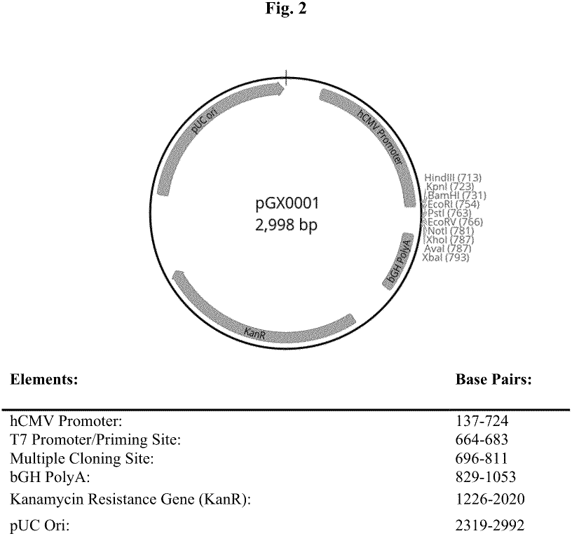

The map and description of the modified expression vector pVAX1 (pGX0001) are shown in FIG. 2. Modifications were introduced into pVAX1 to create pGX0001 and are identified based on the reported sequence of pVAX1 available from Thermo Fisher Scientific. These modifications are:

| | | |
|---|---|---|
| C>G | 241 | in hCMV promoter |
| C>T | 1158 | backbone, downstream of the bovine growth hormone polyadenylation signal (bGHpolyA) |
| A> - | 2092 | backbone, downstream of the Kanamycin resistance gene (KanR) |
| C>T | 2493 | in pUC origin of replication (pUC ori) |
| G>C | 2969 | in very end of pUC Ori upstream of RNASeH site |

Base pairs 2, 3 and 4 are changed from ACT to CTG in backbone, upstream of hCMV promoter.

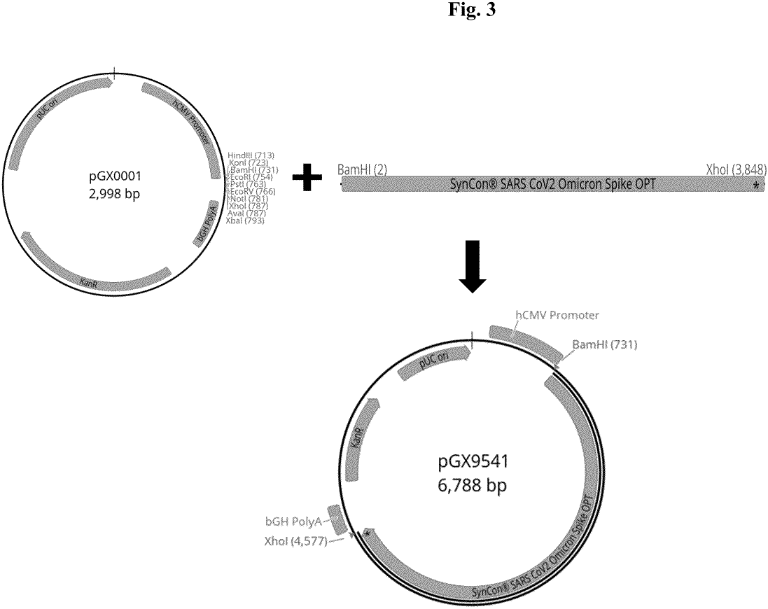

As shown in FIG. 3, pGX9541 was made by cloning of the SynCon® SARS-CoV-2 Omicron Spike OPT Coding Sequence into pGX0001 at the BamHI and XhoI sites. The DNA sequence for the SynCon® SARS-CoV-2 Omicron Spike OPT Coding Sequence was generated to reflect the amino acid changes observed in the Omicron SARS CoV2 variant and then optimized using Inovio’s proprietary Gene Optimization ALgorithm (GOAL) for expression in humans.

Data on SARS-CoV-2 genome sequence submissions was collected using GISAID (Global Initiative on Sharing All Influenza Data). All sequence entries (176 sequence entries) designated to lineage B.1.1.529 that were available at the time of design were collected from GISAID (Shu et al, 2017, EuroSurveillance, 22(13) doi: 10.2807/1560-7917.ES.2017.22.13.30494 PMCID: PMC5388101; Rambaut et al, 2020, Nat Microbiol. 2020 Nov;5(11):1403-1407. doi: 10.1038/s41564-020-0770-5. Epub 2020 Jul 15. PMID: 32669681). The accession numbers of entries used in the analysis are available below:

| | | | |
|---|---|---|---|
| EPI_ISL_6590782, | EPI_ISL_6640916, | EPI_ISL_6640917, | EPI_ISL_6640919, |
| EPI_ISL_6647956, | EPI_ISL_6647957, | EPI_ISL_6647958, | EPI_ISL_6647959, |
| EPI_ISL_6647960, | EPI_ISL_6647961, | EPI_ISL_6647962, | EPI_ISL_6670244, |
| EPI_ISL_6698790, | EPI_ISL_6698792, | EPI_ISL_6699728, | EPI_ISL_6699729, |
| EPI_ISL_6699730, | EPI_ISL_6699731, | EPI_ISL_6699732, | EPI_ISL_6699733, |
| EPI_ISL_6699734, | EPI_ISL_6699735, | EPI_ISL_6699736, | EPI_ISL_6699737, |
| EPI_ISL_6699738, | EPI_ISL_6699739, | EPI_ISL_6699740, | EPI_ISL_6699741, |
| EPI_ISL_6699742, | EPI_ISL_6699743, | EPI_ISL_6699744, | EPI_ISL_6699745, |
| EPI_ISL_6699746, | EPI_ISL_6699747, | EPI_ISL_6699748, | EPI_ISL_6699749, |
| EPI_ISL_6699750, | EPI_ISL_6699751, | EPI_ISL_6699752, | EPI_ISL_6699753, |
| EPI_ISL_6699754, | EPI_ISL_6699755, | EPI_ISL_6699756, | EPI_ISL_6699757, |
| EPI_ISL_6699758, | EPI_ISL_6699759, | EPI_ISL_6699760, | EPI_ISL_6699761, |
| EPI_ISL_6699762, | EPI_ISL_6699763, | EPI_ISL_6699764, | EPI_ISL_6699765, |
| EPI_ISL_6699766, | EPI_ISL_6699767, | EPI_ISL_6699768, | EPI_ISL_6699769, |
| EPI_ISL_6699770, | EPI_ISL_6699771, | EPI_ISL_6704864, | EPI_ISL_6704867, |
| EPI_ISL_6704870, | EPI_ISL_6704871, | EPI_ISL_6704872, | EPI_ISL_6704873, |
| EPI_ISL_6704874, | EPI_ISL_6704875, | EPI_ISL_6752026, | EPI_ISL_6752027, |
| EPI_ISL_6774081, | EPI_ISL_6774082, | EPI_ISL_6774083, | EPI_ISL_6774084, |
| EPI_ISL_6774085, | EPI_ISL_6774086, | EPI_ISL_6774087, | EPI_ISL_6774088, |
| EPI_ISL_6774089, | EPI_ISL_6774090, | EPI_ISL_6774091, | EPI_ISL_6774092, |
| EPI_ISL_6774093, | EPI_ISL_6777160, | EPI_ISL_6782043, | EPI_ISL_6782055, |
| EPI_ISL_6782056, | EPI_ISL_6782071, | EPI_ISL_6782079, | EPI_ISL_6782080, |
| EPI_ISL_6782084, | EPI_ISL_6782090, | EPI_ISL_6782092, | EPI_ISL_6794907, |
| EPI_ISL_6795188, | EPI_ISL_6795189, | EPI_ISL_6795190, | EPI_ISL_6795191, |
| EPI_ISL_6795192, | EPI_ISL_6795193, | EPI_ISL_6795195, | EPI_ISL_6795199, |
| EPI_ISL_6795202, | EPI_ISL_6795212, | EPI_ISL_6795406, | EPI_ISL_6795833, |
| EPI_ISL_6795835, | EPI_ISL_6795836, | EPI_ISL_6795837, | EPI_ISL_6795838, |
| EPI_ISL_6795839, | EPI_ISL_6795840, | EPI_ISL_6795841, | EPI_ISL_6795842, |
| EPI_ISL_6795844, | EPI_ISL_6795845, | EPI_ISL_6795846, | EPI_ISL_6795847, |
| EPI_ISL_6795848, | EPI_ISL_6795849, | EPI_ISL_6795850, | EPI_ISL_6810482, |
| EPI_ISL_6810483, | EPI_ISL_6810484, | EPI_ISL_6810485, | EPI_ISL_6810487, |
| EPI_ISL_6814922, | EPI_ISL_6814923, | EPI_SL_6818284, | EPI_ISL_6821008, |
| EPI_ISL_6825389, | EPI_ISL_6825390, | EPI_ISL_6825391, | EPI_ISL_6825392, |
| EPI_ISL_6825393, | EPI_ISL_6825394, | EPI_ISL_6825395, | EPI_ISL_6825396, |
| EPI_ISL_6825397, | EPI_ISL_6825398, | EPI_ISL_6825546, | EPI_ISL_6825551, |
| EPI_ISL_6826713, | EPI_ISL_6826714, | EPI_ISL_6829575, | EPI_ISL_6829577, |
| EPI_ISL_6832108, | EPI_ISL_6832737, | EPI_ISL_6833884, | EPI_ISL_6834467, |
| EPI_ISL_6841607, | EPI_ISL_6841608, | EPI_ISL_6841609, | EPI_ISL_6841610, |
| EPI_ISL_6841611, | EPI_ISL_6841612, | EPI_ISL_6841613, | EPI_ISL_6841614, |
| EPI_ISL_6841615, | EPI_ISL_6841616, | EPI_ISL_6841617, | EPI_ISL_6841619, |
| EPI_ISL_6841980, | EPI_ISL_6841981, | EPI_ISL_6842154, | EPI_ISL_6842155, |
| EPI_ISL_6842156, | EPI_ISL_6842157, | EPI_ISL_6842158, | EPI_ISL_6842159, |
| EPI_ISL_6842160, | EPI_ISL_6842161, | EPI_ISL_6842162, | EPI_ISL_6842163, |
| EPI_ISL_6842164, | EPI_ISL_6842165, | EPI_ISL_6842166, | EPI_ISL_6842167 |

Data on mutations observed in the SARS-CoV-2 Spike sequences from these entries was analyzed. The results were then aggregated to determine a common consensus set of mutations observed consistently in these SARS-CoV-2 Spike protein sequences designated to the lineage B.1.1.529. These amino acid changes were then placed in the spike peptide sequence encoded by SynCon® SARS-CoV-2 Spike of pGX9501, the plasmid used in INO-4800, thus generating a SARS-CoV-2 lineage B.1.1.529 (Omicron) specific spike protein sequence.

In addition, a tandem proline mutation (K986P/V987P) named “2P” was added to the SynCon®SARS-CoV-2 Omicron Spike OPT. Also, an IgE leader sequence identical to that used in INO-4800 also replaced the endogenous SARS-CoV-2 Spike signal peptide sequence.

The peptide sequence with the above-mentioned variations was then used as an input in the GOAL algorithm to generate a human expression optimized DNA sequence of SynCon® SARS-CoV-2 Omicron Spike OPT, the coding sequence of pGX9541 (SEQ ID NO: 2).

Included in the construct synthesis was the addition of a Kozak sequence (GCCACC) immediately 5′ of the start codon, in addition to restriction sites for subcloning of the construct into pGX0001 vector (5′ BamHI and 3′ XhoI). The optimized DNA sequence was synthesized, digested with BamHI and XhoI, and cloned into the expression vector pGX0001 under the control of the cytomegalovirus immediate-early promoter.

The amino acid and nucleotide sequence for the SynCon® SARS-CoV-2 Omicron Spike OPT are SEQ ID NO: 1 and SEQ ID NO: 2, respectively.

### Example 2: Immunogenicity of INO-4803, SARS-CoV-2 Omicron Spike-Matched Synthetic DNA Vaccine, in Mice and Hamster Models

### A. Immunogenicity of INO-4803, SARS-CoV-2 Omicron Spike-Matched Synthetic DNA Vaccine, in BALB/c Mice

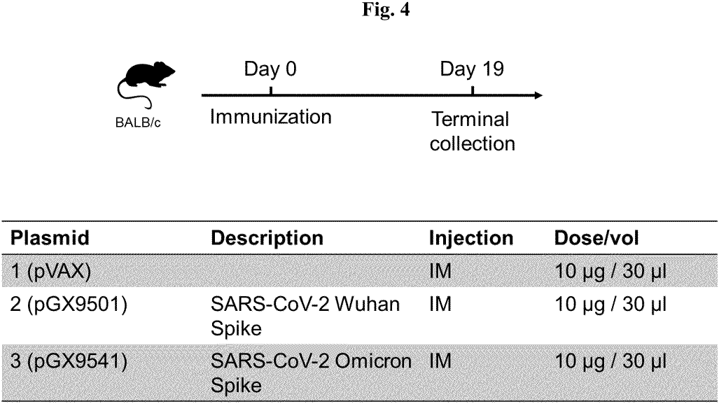

The immunogenicity of DNA vaccine candidate pGX9541 plasmids encoding the SARS-CoV-2 Omicron BA. 1 Spike variant antigen was assessed after administration by intramuscular (IM) injection followed by electroporation (EP) in female BALB/c mice. The study design is outlined in FIG. 4. On day 0 mice received a single dose of DNA plasmid delivered by intramuscular injection followed by electroporation with CELLECTRA® 3P. On day 19, mice were euthanized, and IFNγ ELISpot, antibody binding and pseudovirus neutralization assays were performed on the collected samples.

### Materials & Methods

Binding Antibody ELISA: 96-well high binding Coming Costar half-area plates (cat. no. 3690) were coated with 1 µg/ml of SARS-CoV-2 Spike protein (Acro Biosystems cat. no. SPN-C52H9), 1 µg/ml of SARS-CoV-2 Omicron Variant Spike protein (Acro Biosystems cat. no. SPN-C52Hz), 1 µg/ml of SARS-CoV-2 Delta Variant spike protein (Acro Biosystems cat. no. SPN-C52He). In addition, plates were also coated with 1 µg/ml of SARS-CoV-2 Spike RBD protein (Sino Biological cat. no. 40592-V08B) and 1 µg/ml of SARS-CoV-2 Omicron Variant Spike RBD protein (Sino Biological cat. no. 40592-V08H121). All the proteins contained His tag. All plates were coated in 1x DPBS (Thermo Scientific) overnight at 4° C. The next day, plates were washed with 1x PBS + 0.05% Tween-20 and blocked with 3% BSA in PBS + Tween-20 for 2 hours at room temperature (RT). Plates were then washed as before and serially diluted sera samples were added, and the plates were incubated for 2 hours at RT. Plates were washed and incubated with a 1:10,000 dilution of anti-mouse IgG HRP secondary antibody (Sigma cat. A4416) for 1 hour at room temperature. The plates were washed and 100 µl/well of SureBlue TMB Substrate (KPL 5120-0077) was added to the plates. The reaction was stopped upon the addition of TMB Stop Solution (KPL 5150-0021) after a 10-minute incubation and the plates were read on a Biotek Synergy plate reader at the 450 nm wavelength.

Mouse IFNγ ELISpot Assay: Mouse IFN-γ ELISpot kits were purchased from MabTech (MabTech #3321-4APW-10) to evaluate antigen specific responses. The precoated 96-well plates were washed in PBS according to the manufacturer’s protocol and blocked for at least one hour with R10 media at the room temperature. Isolated splenocytes were resuspended in R10 media and plated in triplicate at 2×10⁵ cells per well. Overlapping 15-mer peptides (overlapping by 9 residues) spanning the full-length spike protein sequence for WT, Delta, or Omicron SARS-2-CoV S protein (Genscript) were used as recall antigens. These peptides were resuspended in DMSO (Sigma) and pooled at a concentration of ~5 µg/ml per peptide into 5 peptide pools for cell stimulation. To make Megapools, each peptide pool was mixed together with same molar equivalent for a final concentration of 1 µg/ml. As a positive control, Concavalin A (Sigma) was used at 5 µg/ml and complete media with DMSO was used as a negative control. The plates were incubated for a minimum of 18 hours at 37° C. 5% CO₂. For development, plates were first washed in PBS then incubated with a biotinylated anti-mouse IFN-γ detection antibody (R4-6A2-biotin) for 2 hours at room temperature. After washing, plates were then incubated with streptavidin-ALP (MabTech #3321-4APW-10) for 1 hour at room temperature. Plates were washed, and spots were detected using a filtered substrate solution (BCIP/NBT-plus) according to manufacturer’s instruction (MabTech). Once the plates were dried, the spots were counted using an automated ELISpot reader (Cellular Technology). The average spot-forming unit (SFU) was adjusted to 1×10⁶ splenocytes, and antigen-specific responses are reported as the number of IFN-γ SFU per 1×10⁶ splenocytes greater than DMSO control.

### SARS-CoV-2 Pseudovirus Neutralization Assay

Pseudovirus production: SARS-CoV-2 pseudovirus stocks encoding for the Wuhan (WT), B.1.617.2 (Delta) B.1.1.529 (Omicron) spike proteins were produced using HEK293T cells transfected with Lipofectamine 3000 (ThermoFisher) using IgE-SARS-CoV-2 S plasmid variants (Genscript) co-transfected with pNL4-3.Luc.R-E- plasmid (NIH AIDS reagent) at a 1:8 ratio, respectively. 72h post transfection, supernatants were collected, steri-filtered (Millipore Sigma), and aliquoted for storage at -80° C.

Pseudovirus neutralization: Chinese hamster ovary (CHO) cells stably expressing ACE2 (ACE2-CHOs) were used as target cells at 7,000 cells/well. SARS-CoV-2 pseudovirus were tittered to yield greater than 30 times the cells only control relative luminescence units (RLU) after 72 h of infection. Animal sera were heat inactivated and serially diluted two-folds starting at 1:32 dilution. Sera were incubated with SARS-CoV-2 pseudovirus for 90 min at room temperature. After incubation, sera-pseudovirus mixture was added to ACE2-CHOs and allowed to incubate in a standard incubator (37° C., 5% CO₂) for 72 h. After 72 h, cells were lysed using Britelite plus Reporter Gene (PerkinElmer®) and RLU was measured using an automated luminometer. Neutralization titers (ID₅₀) were calculated using GraphPad Prism 8 and defined as the reciprocal serum dilution at which RLU were reduced by 50% compared to RLU in virus control wells after subtraction of background RLU in cell control wells.

### Results

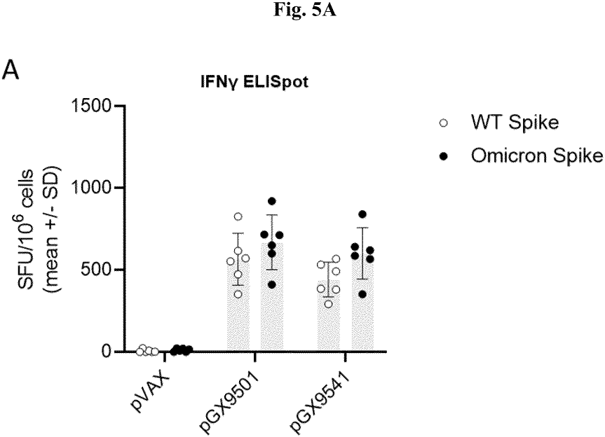

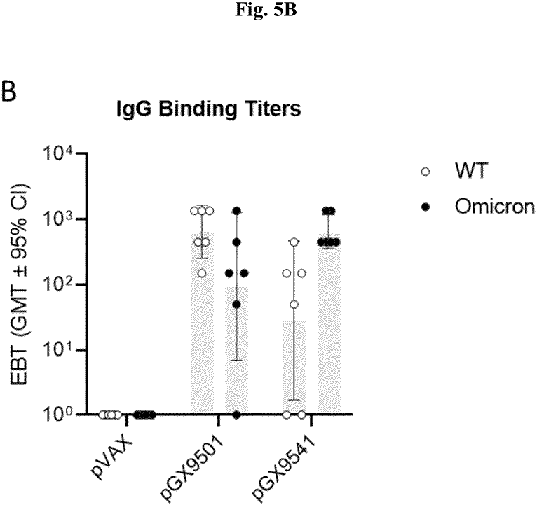

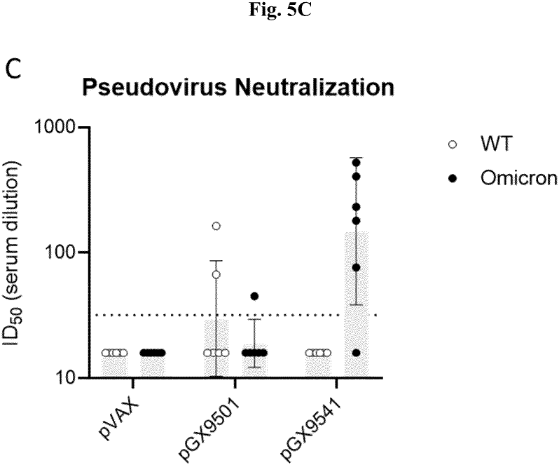

T cell responses were measured on day 19 post single immunization by IFNγ ELISpot after splenocyte stimulation with SARS-CoV-2 peptides. Splenocytes were stimulated with cell culture media containing peptide megapools spanning the WT or Omicron variant spike proteins (FIG. 5A). Antigen-specific T cell responses were detected in mice following a single immunization with 10 µg of SARS-CoV-2 plasmids pGX9501 and pGX9541, but not empty vector control. Immunization with either pGX9501 or pGX9541 resulted in mean spot counts against WT and Omicron Spike peptides that were increased over pGX0001-dosed mice. All test plasmids elicited SARS-CoV-2 spike specific T-cell responses against WT and Omicron spike peptides.

Humoral responses against full-length WT and Omicron SARS-CoV-2 spike protein were measured by binding IgG ELISA in serum samples collected on day 19 post immunization (FIG. 5B). Antibody binding titers were absent in all mice dosed with pGX0001 for all variants tested. Mice immunized with pGX9501 (INO-4800) demonstrated elevated endpoint binding titers compared to control mice, with seroconversion observed in 6/6 and ⅚ animals against the full-length WT and Omicron spike proteins. Antibody binding titers in mice receiving pGX9541 demonstrated 100% seroconversion against the Omicron antigen, and 4/6 animlas seroconverting against the WT antigen.

Pseudovirus neutralization of SARS-CoV-2 Spike variants including the WT and Omicron variants was assessed 19 days post immunization (FIG. 5C). ID₅₀ values were calculated based on the serum dilution that confers 50% inhibitory effect against each pseudovirus variant tested. Immunization with pGX0001 resulted in no neutralizing activity against either pseudovirus. Immunization with pGX9501 resulted in neutralization against the WT pseudovirus in 2 of 6 mice and against Omicron in 1 of 6 mice. Immunization with pGX9541 resulted in neutralization of the Omicron pseudovirus in 5 of 6 mice (FIG. 5C). No cross-neutralizing activity was observed against the WT pseudovirus in mice immunized with pGX9541.

### B: Immunogenicity of INO-4803, SARS-CoV-2 Omicron Spike-Matched Synthetic DNA vAccine, in C57BL/6 Mice

### Materials & Methods

Binding Antibody ELISA: 96-well high binding Corning Costar half-area plates (cat. no. 3690) were coated with 1 µg/ml of SARS-CoV-2 Spike protein (Acro Biosystems cat. no. SPN-C52H9), 1 µg/ml of SARS-CoV-2 Omicron Variant Spike protein (Acro Biosystems cat. no. SPN-C52Hz), 1 µg/ml of SARS-CoV-2 Delta Variant spike protein (Acro Biosystems cat. no. SPN-C52He). In addition, plates were also coated with 1 µg/ml of SARS-CoV-2 Spike RBD protein (Sino Biological cat. no. 40592-V08B) and 1 µg/ml of SARS-CoV-2 Omicron Variant Spike RBD protein (Sino Biological cat. no. 40592-V08H121). All the proteins contained His tag. All plates were coated in 1x DPBS (Thermo Scientific) overnight at 4° C. The next day, plates were washed with 1x PBS + 0.05% Tween-20 and blocked with 3% BSA in PBS + Tween-20 for 2 hours at room temperature (RT). Plates were then washed as before and serially diluted sera samples were added, and the plates were incubated for 2 hours at RT. Plates were washed and incubated with a 1:10,000 dilution of anti-mouse IgG HRP secondary antibody (Sigma cat. A4416) for 1 hour at room temperature. The plates were washed and 100 µl/well of SureBlue TMB Substrate (KPL 5120-0077) was added to the plates. The reaction was stopped upon the addition of TMB Stop Solution (KPL 5150-0021) after a 10-minute incubation and the plates were read on a Biotek Synergy plate reader at the 450 nm wavelength.

Mouse IFNγ ELISpot Assay: Mouse IFN-γ ELISpot kits were purchased from MabTech (MabTech #3321-4APW-10) to evaluate antigen specific responses. The precoated 96-well plates were washed in PBS according to the manufacturer’s protocol and blocked for at least one hour with R10 media at the room temperature. Isolated splenocytes were resuspended in R10 media and plated in triplicate at 2×10⁵ cells per well. Overlapping 15-mer peptides (overlapping by 9 residues) spanning the full-length spike protein sequence for WT, Delta, or Omicron SARS-2-CoV S protein (Genscript) were used as recall antigens. These peptides were resuspended in DMSO (Sigma) and pooled at a concentration of ~5 µg/ml per peptide into 5 peptide pools for cell stimulation. To make Megapools, each peptide pool was mixed together with same molar equivalent for a final concentration of 1 µg/ml. As a positive control, Concavalin A (Sigma) was used at 5 µg/ml and complete media with DMSO was used as a negative control. The plates were incubated for a minimum of 18 hours at 37° C. 5% CO₂. For development, plates were first washed in PBS then incubated with a biotinylated anti-mouse IFN-γ detection antibody (R4-6A2-biotin) for 2 hours at room temperature. After washing, plates were then incubated with streptavidin-ALP (MabTech #3321-4APW-10) for 1 hour at room temperature. Plates were washed, and spots were detected using a filtered substrate solution (BCIP/NBT-plus) according to manufacturer’s instruction (MabTech). Once the plates were dried, the spots were counted using an automated ELISpot reader (Cellular Technology). The average spot-forming unit (SFU) was adjusted to 1×10⁶ splenocytes, and antigen-specific responses are reported as the number of IFN-γ SFU per 1×10⁶ splenocytes greater than DMSO control.

### SARS-CoV-2 Pseudovirus Neutralization Assay

Pseudovirus production: SARS-CoV-2 pseudovirus stocks encoding for the Wuhan (WT), B.1.617.2 (Delta) B.1.1.529 (Omicron) spike proteins were produced using HEK293T cells transfected with Lipofectamine 3000 (ThermoFisher) using IgE-SARS-CoV-2 S plasmid variants (Genscript) co-transfected with pNL4-3.Luc.R-E- plasmid (NIH AIDS reagent) at a 1:8 ratio, respectively. 72h post transfection, supernatants were collected, steri-filtered (Millipore Sigma), and aliquoted for storage at -80° C.

Pseudovirus neutralization: Chinese hamster ovary (CHO) cells stably expressing ACE2 (ACE2-CHOs) were used as target cells at 7,000 cells/well. SARS-CoV-2 pseudovirus were tittered to yield greater than 30 times the cells only control relative luminescence units (RLU) after 72h of infection. Animal sera were heat inactivated and serially diluted two-folds starting at 1:32 dilution. Sera were incubated with SARS-CoV-2 pseudovirus for 90 min at room temperature. After incubation, sera-pseudovirus mixture was added to ACE2-CHOs and allowed to incubate in a standard incubator (37° C., 5% CO₂) for 72h. After 72h, cells were lysed using Britelite plus Reporter Gene (PerkinElmer®) and RLU was measured using an automated luminometer. Neutralization titers (ID₅₀) were calculated using GraphPad Prism 8 and defined as the reciprocal serum dilution at which RLU were reduced by 50% compared to RLU in virus control wells after subtraction of background RLU in cell control wells.

### Results:

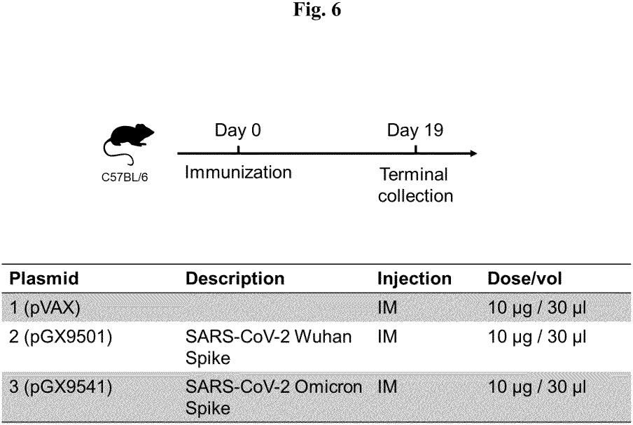

The immunogenicity of DNA vaccine candidate pGX9541 plasmid encoding the SARS-CoV-2 Omicron BA. 1 Spike variant antigen was assessed after administration by intramuscular (IM) injection followed by electroporation (EP) in female C57BL/6 mice. The study design is outlined in FIG. 6. On day 0, mice received a single dose of DNA plasmid delivered by intramuscular injection followed by electroporation with CELLECTRA 3P. On day 19, mice were euthanized, and IFNγ ELISpot, antibody binding and pseudovirus neutralization assays were performed on the collected samples.

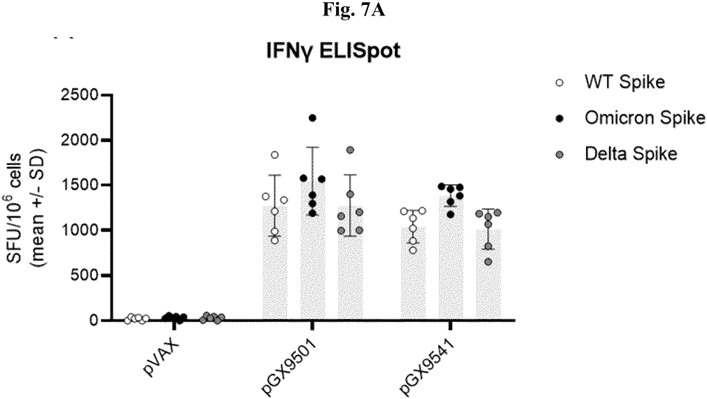

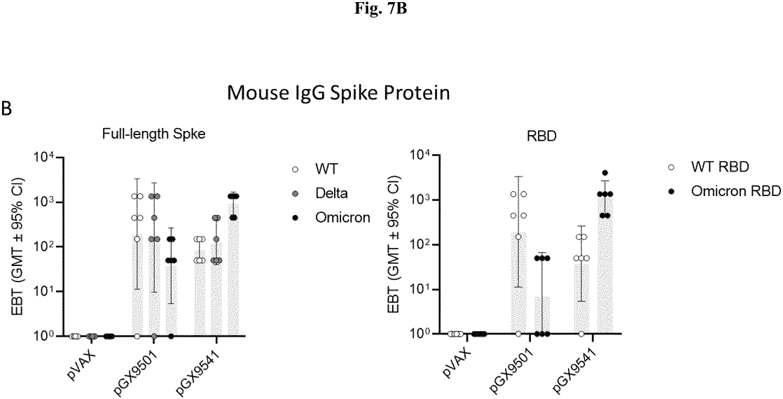

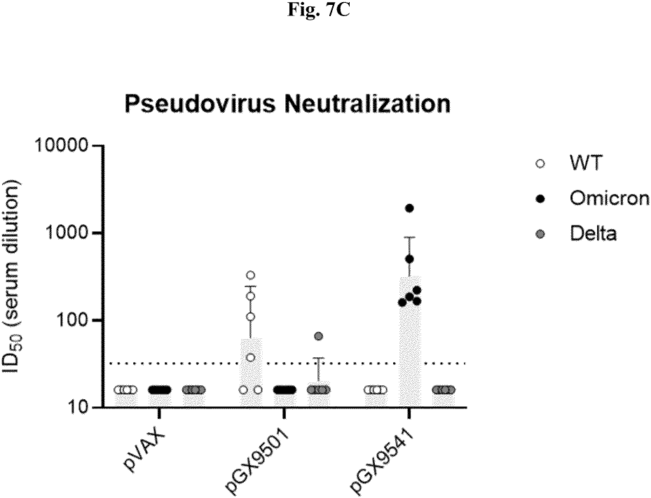

T cell responses were measured on day 19 post immunization by IFNγ ELISpot after splenocyte stimulation with SARS-CoV-2 peptides. Splenocytes were stimulated with cell culture media containing peptide megapools spanning the WT, Delta, or Omicron variant spike proteins (FIG. 7A). Antigen-specific T cell responses were detected in mice following a single immunization with 10 µg of either pGX9501 or pGX9541, but not in mice receiving the empty vector control. Immunization with either pGX9501 or pGX9541 resulted in mean spot counts against WT, Delta, and Omicron peptides that were increased compared to pGX0001-dosed mice. All test plasmids elicited robust SARS-CoV-2 spike specific T-cell responses against WT, Delta, and Omicron spike peptides.

Humoral responses against the full-length WT, Delta, and Omicron SARS-CoV-2 spike proteins were measured by binding IgG ELISA in serum samples collected on day 19 post immunization (FIG. 7B). Antibody binding titers were absent in all mice dosed with pGX0001 for all variants tested. Mice immunized with pGX9501 demonstrated elevated antibody binding titers compared to mice receiving pGX0001, with seroconversion observed in ⅚ animals against all variants. Mice immunized with pGX9541 showed broad antibody responses across all variants tested, with all animals demonstrating antibody responses against WT, Delta and Omicron.

Compared to WT, Omicron has acquired 15 amino acid substitutions in the RBD alone. Since the RBD is the region within the Spike responsible for binding to the ACE2 cell receptor, we further investigated the antibody binding responses in groups treated with pGX9501 and pGX9541 to WT and Omicron RBD proteins. No binding was observed in the group treated with pGX0001 in all variants tested. In contrast, we detected robust binding to WT RBD and elevated titers to Omicron RBD (3/6 seroconversion) in the group treated with pGX9501. In the group immunized with pGX9541, ⅚ and 6/6 animals showed seroconversion against WT and Omicron RBD proteins, respectively (FIG. 7B).

Pseudovirus neutralization of SARS-CoV-2 Spike variants including the WT, Delta, and Omicron variants were assessed 19 days post single immunization as shown in FIG. 4C. ID50 values were calculated based on the dilution of serum that confers 50% inhibitory effect in each pseudovirus variants tested. Immunization with pGX0001 resulted in no neutralizing activity against any of the variants tested. Immunization with pGX9501 resulted in increased neutralization of WT variant in 4 of 6 mice but reduced neutralizing activity against Delta (⅙ mice) and no activity to Omicron (FIG. 7C). Immunization with pGX9541 resulted in increased neutralizing responses against Omicron with 100% seroconversion. Cross-neutralizing responses against the WT or Delta pseudoviruses were not observed in mice receiving pGX9541.

### C: Immunogenicity of INO-4803, SARS-CoV-2 Omicron Spike-Matched Synthetic DNA Vaccine, in Syrian Golden Hamsters

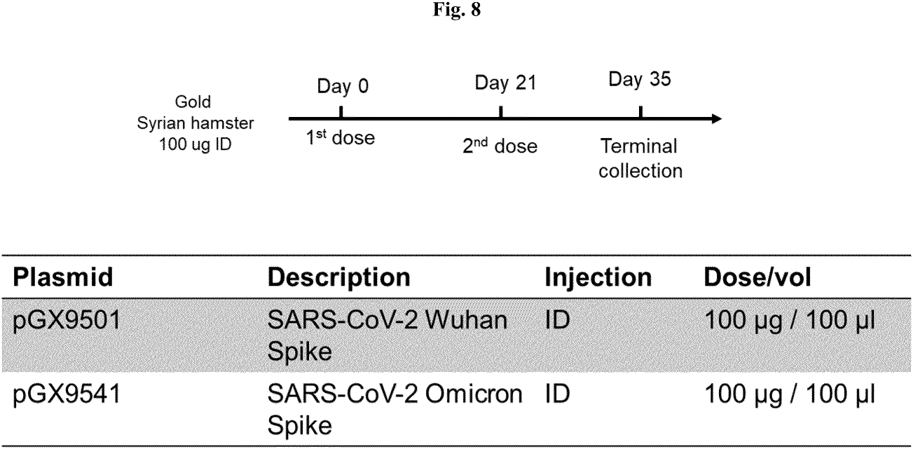

The immunogenicity of DNA vaccine candidate pGX9541 plasmids encoding the SARS-CoV-2 Omicron BA. 1 Spike variant antigen was assessed after administration by intradermal (ID) injection followed by electroporation (EP) in Syrian Golden hamsters. The study design is outlined in FIG. 8. On days 0 and 21, animals received DNA plasmid delivered by intradermal injection followed by electroporation with CELLECTRA® 3P. On day 35, animals were euthanized, and IFNγ ELISpot, and pseudovirus neutralization assays were performed on the collected samples.

### Materials & Methods:

### SARS-CoV-2 Pseudovirus Neutralization Assay

Pseudovirus production: SARS-CoV-2 pseudovirus stocks encoding for the Wuhan (WT), B.1.617.2 (Delta) B.1.1.529 (Omicron) spike proteins were produced using HEK293T cells transfected with Lipofectamine 3000 (ThermoFisher) using IgE-SARS-CoV-2 S plasmid variants (Genscript) co-transfected with pNL4-3.Luc.R-E- plasmid (NIH AIDS reagent) at a 1:8 ratio, respectively. 72h post transfection, supernatants were collected, steri-filtered (Millipore Sigma), and aliquoted for storage at -80° C.

Pseudovirus neutralization: CHO cells stably expressing ACE2 (ACE2-CHOs) were used as target cells at 7,000 cells/well. SARS-CoV-2 pseudovirus were tittered to yield greater than 30 times the cells only control relative luminescence units (RLU) after 72h of infection. Animal sera were heat inactivated and serially diluted two-folds starting at 1:32 dilution. Sera were incubated with SARS-CoV-2 pseudovirus for 90 min at room temperature. After incubation, sera-pseudovirus mixture was added to ACE2-CHOs and allowed to incubate in a standard incubator (37° C., 5% CO₂) for 72 h. After 72 h, cells were lysed using Britelite plus Reporter Gene (PerkinElmer™) and RLU was measured using an automated luminometer. Neutralization titers (ID₅₀) were calculated using GraphPad Prism 8 and defined as the reciprocal serum dilution at which RLU were reduced by 50% compared to RLU in virus control wells after subtraction of background RLU in cell control wells.

Hamster IFN-γ ELISpot: Hamster IFN-γ ELISpot kit was purchased from MabTech (MabTech # 3102-2H) to evaluate antigen specific responses. PVDF 96-well plates (Millipore cat# MSIPS4W10) were activated with 35% Ethanol and coated with hamster IFNγ antibodies following manufacturer’s protocol. Plates were incubated at 4 degrees for 24 h. Next, plates were washed 5x with PBS and blocked for at least one hour with R10 media at the room temperature. Isolated splenocytes were resuspended in R10 media and plated in triplicate at 2×10⁵ cells per well. Overlapping 15-mer peptides (overlapping by 9 residues) spanning the full-length spike protein sequence for WT or Omicron SARS-2-CoV S protein (Genscript) were used as recall antigens. These peptides were resuspended in DMSO (Sigma) and pooled at a concentration of ~5 µg/ml per peptide into 5 peptide pools for cell stimulation. To make Megapools, each peptide pool was mixed together with same molar equivalent for a final concentration of 1 µg/ml. As a positive control, Concavalin A (Sigma) was used at 5 µg/ml and complete media with DMSO was used as a negative control. The plates were incubated for a minimum of 18 hours at 37° C. 5% CO₂. For development, plates were first washed in PBS then incubated with a biotinylated anti-hamster IFN-γ detection antibody for 2 hours at room temperature. After washing, plates were then incubated with streptavidin-ALP for 1 hour at room temperature. Plates were washed, and spots were detected using a filtered substrate solution (BCIP/NBT-plus) according to manufacturer’s instruction (MabTech). Once the plates were dried, the spots were counted using an automated ELISpot reader (Cellular Technology). The average spot-forming unit (SFU) was adjusted to 1×10⁶ splenocytes, and antigen-specific responses are reported as the number of IFN-γ SFU per 1×10⁶ splenocytes greater than DMSO control.

### Results:

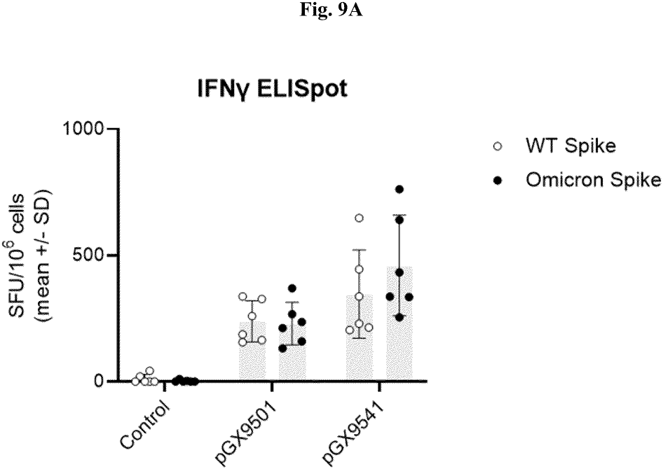

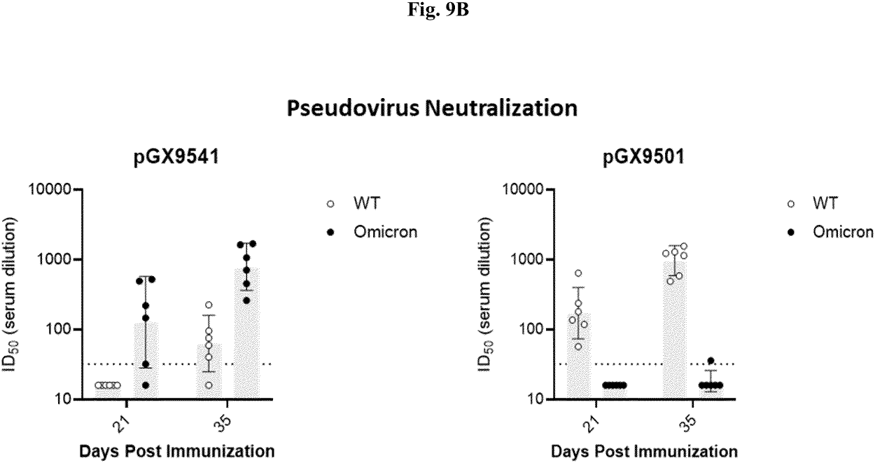

T cell responses were measured on day 35 post second immunization by IFNγ ELISpot after splenocyte stimulation with SARS-CoV-2 peptides. Splenocytes were stimulated with cell culture media containing peptide megapools spanning the WT or Omicron variant spike proteins (FIG. 9A). Antigen-specific T cell responses were detected in hamsters following two immunizations with 100 µg of SARS-CoV-2 plasmids pGX9501 and pGX9541, but not in the naïve group. All test plasmids elicited SARS-CoV-2 spike specific T-cell responses against WT and Omicron spike peptides.

Pseudovirus neutralization of SARS-CoV-2 Spike variants including the WT and Omicron variants were assessed 21 and 35 days post first or second immunizations, respectively (FIG. 9B). ID₅₀ values were calculated based on the dilution of serum that confers 50% inhibitory effect in each pseudovirus variants tested. Immunization with pGX9501 resulted in increased neutralization of WT on both days 21 and 35, with an increase in ID₅₀ titers after two immunizations. pGX9501 generated little to no neutralizing activity against the Omicron pseudovirus. Immunization with pGX9541 resulted in neutralization of the Omicron pseudovirus in 5 of 6 hamsters after single immunization. After two immunizations, all pGX9541-immunized hamsters showed neutralizing activity to the Omicron pseudovirus. In addition, pGX9541 treatment induced cross-neutralizing antibodies to WT pseudovirus after two immunizations. No neutralizing activity was observed in the control group.

### Conclusion

Treatment with pGX9541 was shown to be immunogenic in mice (BALB/c, C57BL/6) and hamster (Syrian Golden hamster) models by eliciting humoral and T cell responses to Omicron spike. In addition, pGX9541 demonstrated cross-reactive antibody and T cell responses against all spike proteins tested, including WT, Delta and Omicron.

Treatment with pGX9541 elicited neutralizing antibodies against the Omicron pseudovirus after single immunization in mice as well as hamsters. In mice immunized with pGX9541, neutralizing antibodies against WT and Delta were low or absent. In contrast, mice immunized with pGX9501 showed an increase in neutralizing response against the WT pseduovirus, but no neutralization against Omicron. In contrast, hamsters immunized with pGX9541 showed an increase in neutralizing antibodies to WT after two immunizations. All hamsters treated with pGX9541 elicited neutralizing antibodies to Omicron after single immunization and an increase in response after two immunizations.

Cellular responses as measured by the levels of IFNγ were detected against all spike peptides tested in all groups receiving the Omicron plasmid vaccines as well as in the group receiving pGX9501. This finding demonstrates the ability of SARS-CoV-2 spike plasmid DNA vaccines to retain cellular responses across multiple variants.

Altogether, treatment with pGX9541 can induce both humoral and T cell responses to Omicron Spike after single immunization. In addition, pGX9541 induces cross T-cell immunity to the ancestral WT SARS-CoV-2 as well as cross-neutralizing antibodies to WT after two immunizations.

### SEQUENCES

### SEQ ID NO: 1 - Amino Acid Sequence of SARS CoV2 Omicron Spike OPT (SARS-CoV-2 Consensus Spike Antigen Amino Acid Sequence Encoded by Antigen Insert Sequence of pGX9541) (IgE Leader Sequence Underlined)

1 MDWTWILFLV AAATRVHSSQ CVNLTTRTQL PPAYTNSFTR GVYYPDKVFR SSVLHSTQDL
61 FLPFFSNVTW FHVISGTNGT KRFDNPVLPF NDGVYFASIE KSNIIRGWIF GTTLDSKTQS
121 LLIVNNATNV VIKVCEFQFC NDPFLDHKNN KSWMESEFRV YSSANNCTFE YVSQPFLMDL
181 EGKQGNFKNL REFVFKNIDG YFKIYSKHTP IIVREPEDLP QGFSALEPLV DLPIGINITR
241 FQTLLALHRS YLTPGDSSSG WTAGAAAYYV GYLQPRTFLL KYNENGTITD AVDCALDPLS
301 ETKCTLKSFT VEKGIYQTSN FRVQPTESIV RFPNITNLCP FDEVFNATRF ASVYAWNRKR
361 ISNCVADYSV LYNLAPFFTF KCYGVSPTKL NDLCFTNVYA DSFVIRGDEV RQIAPGQTGN
421 IADYNYKLPD DFTGCVIAWN SNKLDSKVSG NYNYLYRLFR KSNLKPFERD ISTEIYQAGN
481 KPCNGVAGFN CYFPLRSYSF RPTYGVGHQP YRVVVLSFEL LHAPATVCGP KKSTNLVKNK
541 CVNFNFNGLK GTGVLTESNK KFLPFQQFGR DIADTTDAVR DPQTLEILDI TPCSFGGVSV
601 ITPGTNTSNQ VAVLYQGVNC TEVPVAIHAD QLTPTWRVYS TGSNVFQTRA GCLIGAEYVN
661 NSYECDIPIG AGICASYQTQ TKSHRRARSV ASQSIIAYTM SLGAENSVAY SNNSIAIPTN
721 FTISVTTEIL PVSMTKTSVD CTMYICGDST ECSNLLLQYG SFCTQLKRAL TGIAVEQDKN
781 TQEVFAQVKQ IYKTPPIKYF GGFNFSQILP DPSKPSKRSF IEDLLFNKVT LADAGFIKQY
841 GDCLGDIAAR DLICAQKFKG LTVLPPLLTD EMIAQYTSAL LAGTITSGWT FGAGAALQIP
901 FAMQMAYRFN GIGVTQNVLY ENQKLIANQF NSAIGKIQDS LSSTASALGK LQDVVNHNAQ
961 ALNTLVKQLS SKFGAISSVL NDIFSRLDPP EAEVQIDRLI TGRLQSLQTY VTQQLIRAAE
1021 IRASANLAAT KMSECVLGQS KRVDFCGKGY HLMSFPQSAP HGVVFLHVTY VPAQEKNFTT
1081 APAICHDGKA HFPREGVFVS NGTHWFVTQR NFYEPQIITT DNTFVSGNCD VVIGIVNNTV
1141 YDPLQPELDS FKEELDKYFK NHTSPDVDLG DISGINASVV NIQKEIDRLN EVAKNLNESL
1201 IDLQELGKYE QYIKWPWYIW LGFIAGLIAI VMVTIMLCCM TSCCSCLKGC CSCGSCCKFD
1261 EDDSEPVLKG VKLHYT**

### SEQ ID NO: 2 - Nucleic Acid Sequence Encoding SARS CoV2 Omicron Spike OPT (SARS-CoV-2 Consensus Spike Antigen Insert Sequence of pGX9541) (IgE Leader Sequence Underlined)

1 atggattgga cctggattct ctttcttgtq gccgcagcaa cccgagtgca ctcaagccag
61 tgtgtgaacc tgaccacaag aactcagctg cctcctgcct acaccaacag cttcaccaga
121 ggagtctact acccagacaa ggtgttcaga tcttctgtgc tgcacagcac ccaggacctc
181 ttcctgccct tcttcagcaa cgtgacctgg ttccacgtga tctctggcac caacggcacc
241 aagagatttg acaaccctgt gctgcctttt aatgatggtg tgtactttgc cagcatcgag
301 aagagcaaca tcatccgagg ctggatcttt ggcaccaccc tggacagcaa gacccagagc
361 ctgctgatcg tgaacaatgc caccaacgtg gtcatcaagg tgtgtgagtt ccagttctgc
421 aatgaccctt tcctggacca caagaacaac aagtcctgga tggaatctga atttagagtg
481 tacagctctg ccaacaactg cacatttgaa tatgtgtccc agcctttcct gatggacctg
541 gagggcaaac aaggcaactt caagaacctg agagaatttg tgttcaagaa cattgatggc
601 tacttcaaga tctacagcaa gcacacaccc atcatcgtga gagaacctga agatcttcct
661 cagggcttca gcgccctgga gcctctggtg gacctgccca tcggcatcaa catcaccagg
721 ttccagaccc tgctggccct gcacagaagc tacctgacac ctggagacag cagcagcggc
781 tggactgctg gagctgctgc ctactacgtg ggctacctgc agccaagaac cttcctgctg
841 aagtacaacg aaaatggcac catcacagat gctgttgact gtgccctgga ccctctttct
901 gagaccaagt gcaccctgaa gtccttcaca gtggagaagg gcatctacca gaccagcaac
961 tttagagtgc agccaacaga gagcatcgtg agatttccaa acatcaccaa cctgtgccca
1021 tttgatgaag tgttcaatgc cacaagattc gcctctgtgt acgcctggaa cagaaaaaga
1081 atcagcaact gtgtggctga ctactctgtg ctgtacaacc tggccccttt cttcaccttc
1141 aagtgctacg gtgtgtctcc aaccaagctg aatgacctgt gcttcaccaa cgtgtatgct
1201 gacagctttg tcatcagagg agatgaagtg agacaaattg ctcctggcca gacaggcaac
1261 attgctgact acaactacaa acttcctgat gacttcacag gctgtgtcat cgcctggaac
1321 agcaacaagc tggacagcaa agtttctggc aattacaact acctgtacag actcttcaga
1381 aaaagcaacc tgaagccatt tgaaagagac atcagcacag agatctacca ggctggcaac
1441 aagccctgca acggagtggc tggcttcaac tgctacttcc ctctgcgctc ctacagcttc
1501 cggcccacct acggcgtggg ccaccagcct tacagagtgg tggtgctgtc ctttgagctg
1561 ctgcacgccc ctgccacagt gtgtggcccc aagaagagca ccaacctggt gaagaacaaa
1621 tgtgtgaact tcaactttaa tggcctgaag ggcacaggag tgctgacaga gagcaacaag
1681 aagttcctgc ccttccagca gtttggaaga gacattgctg acaccacaga tgctgtgaga
1741 gatcctcaga ccctggagat cctggacatc acaccttgct cctttggagg agtttctgtc
1801 atcacacctg gcaccaacac cagcaaccag gtggctgtgc tgtaccaagg agtcaactgc
1861 acagaagttc ctgtggccat ccatgctgac cagctgaccc caacatggag agtgtacagc
1921 acaggcagca acgtgttcca gacaagagct ggctgcctga ttggagctga gtacgtgaac
1981 aacagctatg aatgtgacat ccctattgga gctggcatct gtgcctccta ccagacccaa
2041 accaagagcc acagaagagc aagatctgtg gccagccaga gcatcatcgc ctacaccatg
2101 agcctgggag ctgaaaattc tgtggcctac agcaataaca gcatcgccat ccccaccaac
2161 ttcaccatct ctgtgaccac agagatcctg cctgtgtcca tgacaaaaac atctgtggac
2221 tgcaccatgt acatctgtgg agacagcaca gaatgcagca acctgctgct gcagtacggc
2281 tccttctgca cccagctgaa gagagccctg acaggaattg ctgtggagca ggacaagaac
2341 acacaggaag tttttgccca ggtgaagcag atctacaaaa caccacccat caagtacttt
2401 ggcggcttca acttctccca gatcctgcct gacccttcca agcccagcaa aagaagcttc
2461 atcgaggacc tgctgttcaa caaggtgacc ctggctgatg ctggcttcat caagcagtat
2521 ggagactgcc tgggagacat tgctgcccgg gacctgatct gtgcccagaa gttcaagggc
2581 ctgacagtgc tgcctcctct gctgacagat gaaatgatcg cccagtacac atctgccctg
2641 ctggctggca ccatcaccag cggctggaca tttggagctg gagctgccct gcagatccct
2701 tttgccatgc agatggccta cagatttaat ggcatcggcg tcacccagaa cgtgctgtat
2761 gaaaaccaga agctgatcgc caaccagttt aacagtgcca tcggcaagat ccaggacagc
2821 ctgagcagca cagcctctgc cctgggcaag ctgcaggatg tggtgaatca caatgcccaa
2881 gccctgaaca ccctggtgaa gcagctgagc agcaaatttg gagccatctc cagcgtgctg
2941 aatgacatct tctccaggct ggaccctcct gaagctgagg tgcagatcga ccgcctgatc
3001 acaggccggc tgcagagcct gcagacctac gtgacccagc agctgatcag agctgctgag
3061 atcagagctt ctgccaacct ggctgccacc aagatgtctg aatgtgtgct gggccagagc
3121 aaaagagtgg acttctgtgg caagggctac cacctgatgt ccttccctca gtctgctcct
3181 cacggcgtgg tgttcctgca cgtgacctac gtgcctgccc aggagaagaa cttcaccaca
3241 gcccctgcca tctgccacga cggcaaggcc cacttcccaa gagaaggcgt ctttgtgtcc
3301 aatggcaccc actggttcgt gacacagaga aacttctacg agcctcagat catcaccaca
3361 gacaacacat ttgtttctgg caactgtgat gtggtcatcg gcatcgtgaa caacacagtt
3421 tatgaccctc tgcagcctga gctggacagc ttcaaggagg agctggacaa gtacttcaag
3481 aaccacacca gccctgatgt ggacctgggc gacatctctg gcatcaatgc ctctgtggtg
3541 aacatccaga aggagatcga caggctgaac gaagtggcca agaatctgaa cgaaagcctc
3601 atcgacctgc aggagctggg caagtacgag cagtacatca agtggccttg gtacatctgg
3661 ctgggcttca tcgccggcct catcgccatc gtgatggtga ccatcatgct gtgctgcatg
3721 accagctgct gctcttgcct gaagggctgc tgcagctgtg gcagctgctg caagtttgat
3781 gaagatgatt ctgagcctgt gctgaagggc gtgaagctgc actacacatg ataa

### SEQ ID NO: 3 - Single Strand DNA Sequence of pGX9541 (Antigen Insert Sequence Underlined)

1 gctgcttcgc gatgtacggg ccagatatac gcgttgacat tgattattga ctagttatta
61 atagtaatca attacggggt cattagttca tagcccatat atggagttcc gcgttacata
121 acttacggta aatggcccgc ctggctgacc gcccaacgac ccccgcccat tgacgtcaat
181 aatgacgtat gttcccatag taacgccaat agggactttc cattgacgtc aatgggtgga
241 gtatttacgg taaactgccc acttggcagt acatcaagtg tatcatatgc caagtacgcc
301 ccctattgac gtcaatgacg gtaaatggcc cgcctggcat tatgcccagt acatgacctt
361 atgggacttt cctacttggc agtacatcta cgtattagtc atcgctatta ccatggtgat
421 gcggttttgg cagtacatca atgggcgtgg atagcggttt gactcacggg gatttccaag
481 tctccacccc attgacgtca atgggagttt gttttggcac caaaatcaac gggactttcc
541 aaaatgtcgt aacaactccg ccccattgac gcaaatgggc ggtaggcgtg tacggtggga
601 ggtctatata agcagagctc tctggctaac tagagaaccc actgcttact ggcttatcga
661 aattaatacg actcactata gggagaccca agctggctag cgtttaaact taagcttggt
721 accgagctcg gatccgccac catggattgg acctggattc tctttcttgt ggccgcagca
781 acccgagtgc actcaagcca gtgtgtgaac ctgaccacaa gaactcagct gcctcctgcc
841 tacaccaaca gcttcaccag aggagtctac tacccagaca aggtgttcag atcttctgtg
901 ctgcacagca cccaggacct cttcctgccc ttcttcagca acgtgacctg gttccacgtg
961 atctctggca ccaacggcac caagagattt gacaaccctg tgctgccttt taatgatggt
1021 gtgtactttg ccagcatcga gaagagcaac atcatccgag gctggatctt tggcaccacc
1081 ctggacagca agacccagag cctgctgatc gtgaacaatg ccaccaacgt ggtcatcaag
1141 gtgtgtgagt tccagttctg caatgaccct ttcctggacc acaagaacaa caagtcctgg
1201 atggaatctg aatttagagt gtacagctct gccaacaact gcacatttga atatgtgtcc
1261 cagcctttcc tgatggacct ggagggcaaa caaggcaact tcaagaacct gagagaattt
1321 gtgttcaaga acattgatgg ctacttcaag atctacagca agcacacacc catcatcgtg
1381 agagaacctg aagatcttcc tcagggcttc agcgccctgg agcctctggt ggacctgccc
1441 atcggcatca acatcaccag gttccagacc ctgctggccc tgcacagaag ctacctgaca
1501 cctggagaca gcagcagcgg ctggactgct ggagctgctg cctactacgt gggctacctg
1561 cagccaagaa ccttcctgct gaagtacaac gaaaatggca ccatcacaga tgctgttgac
1621 tgtgccctgg accctctttc tgagaccaag tgcaccctga agtccttcac agtggagaag
1681 ggcatctacc agaccagcaa ctttagagtg cagccaacag agagcatcgt gagatttcca
1741 aacatcacca acctgtgccc atttgatgaa gtgttcaatg ccacaagatt cgcctctgtg
1801 tacgcctgga acagaaaaag aatcagcaac tgtgtggctg actactctgt gctgtacaac
1861 ctggcccctt tcttcacctt caagtgctac ggtgtgtctc caaccaagct gaatgacctg
1921 tgcttcacca acgtgtatgc tgacagcttt gtcatcagag gagatgaagt gagacaaatt
1981 gctcctggcc agacaggcaa cattgctgac tacaactaca aacttcctga tgacttcaca
2041 ggctgtgtca tcgcctggaa cagcaacaag ctggacagca aagtttctgg caattacaac
2101 tacctgtaca gactcttcag aaaaagcaac ctgaagccat ttgaaagaga catcagcaca
2161 gagatctacc aggctggcaa caagccctgc aacggagtgg ctggcttcaa ctgctacttc
2221 cctctgcgct cctacagctt ccggcccacc tacggcgtgg gccaccagcc ttacagagtg
2281 gtggtgctgt cctttgagct gctgcacgcc cctgccacag tgtgtggccc caagaagagc
2341 accaacctgg tgaagaacaa atgtgtgaac ttcaacttta atggcctgaa gggcacagga
2401 gtgctgacag agagcaacaa gaagttcctg cccttccagc agtttggaag agacattgct
2461 gacaccacag atgctgtgag agatcctcag accctggaga tcctggacat cacaccttgc
2521 tcctttggag gagtttctgt catcacacct ggcaccaaca ccagcaacca ggtggctgtg
2581 ctgtaccaag gagtcaactg cacagaagtt cctgtgqcca tccatgctga ccagctgacc
2641 ccaacatgga gagtgtacag cacaggcagc aacgtgttcc agacaagagc tggctgcctg
2701 attggagctg agtacgtgaa caacagctat gaatgtgaca tccctattgg agctggcatc
2761 tgtgcctcct accagaccca aaccaagagc cacagaagag caagatctgt ggccagccag
2821 agcatcatcg cctacaccat gagcctggga gctgaaaatt ctgtggccta cagcaataac
2881 agcatcgcca tccccaccaa cttcaccatc tctgtgacca cagagatcct gcctgtgtcc
2941 atgacaaaaa catctgtgga ctgcaccatg tacatctgtg gagacagcac agaatgcagc
3001 aacctgctgc tgcagtacgg ctccttctgc acccagctga agagagccct gacaggaatt
3061 gctgtggagc aggacaagaa cacacaggaa gtttttgccc aggtgaagca gatctacaaa
3121 acaccaccca tcaagtactt tggcggcttc aacttctccc agatcctgcc tgacccttcc
3181 aagcccagca aaagaagctt catcgaggac ctgctgttca acaaggtgac cctggctgat
3241 gctggcttca tcaagcagta tggagactgc ctgggagaca ttgctgcccg ggacctgatc
3301 tgtgcccaga agttcaaggg cctgacagtg ctgcctcctc tgctgacaga tgaaatgatc
3361 gcccagtaca catctgccct gctggctggc accatcacca gcggctggac atttggagct
3421 ggagctgccc tgcagatccc ttttgccatg cagatggcct acagatttaa tggcatcggc
3481 gtcacccaga acgtgctgta tgaaaaccag aagctgatcg ccaaccagtt taacagtgcc
3541 atcggcaaga tccaggacag cctgagcagc acagcctctg ccctgggcaa gctgcaggat
3601 gtggtgaatc acaatgccca agccctgaac accctggtga agcagctgag cagcaaattt
3661 ggagccatct ccagcgtgct gaatgacatc ttctccaggc tggaccctcc tgaagctgag
3721 gtgcagatcg accgcctgat cacaggccgg ctgcagagcc tgcagaccta cgtgacccag
3781 cagctgatca gagctgctga gatcagagct tctgccaacc tggctgccac caagatgtct
3841 gaatgtgtgc tgggccagag caaaaqagtg gacttctgtg gcaagggcta ccacctgatg
3901 tccttccctc agtctgctcc tcacggcgtg gtgttcctgc acgtgaccta cgtgcctgcc
3961 caggagaaga acttcaccac agcccctgcc atctgccacg acggcaaggc ccacttccca
4021 agagaaggcg tctttgtgtc caatggcacc cactggttcg tgacacagag aaacttctac
4081 gagcctcaga tcatcaccac agacaacaca tttqtttctg gcaactgtga tgtggtcatc
4141 ggcatcgtga acaacacagt ttatgaccct ctgcagcctg agctggacag cttcaaggag
4201 gagctggaca agtacttcaa gaaccacacc agccctgatg tggacctggg cgacatctct
4261 ggcatcaatg cctctgtggt gaacatccag aaggagatcg acaggctgaa cgaagtggcc
4321 aagaatctga acgaaagcct catcgacctg caggagctgg gcaagtacga gcagtacatc
4381 aagtggcctt ggtacatctg gctgggcttc atcgccggcc tcatcgccat cgtgatggtg
4441 accatcatgc tgtgctgcat gaccagctgc tgctcttgcc tgaagggctg ctgcagctgt
4501 ggcagctgct gcaagtttga tgaagatgat tctgagcctg tgctgaaggg cgtgaagctg
4561 cactacacat gataactcga gtctagaggg cccgtttaaa cccgctgatc agcctcgact
4621 gtgccttcta gttgccagcc atctgttgtt tgcccctccc ccgtgccttc cttgaccctg
4681 gaaggtgcca ctcccactgt cctttcctaa taaaatgagg aaattgcatc gcattgtctg
4741 agtaggtgtc attctattct ggggggtggg gtggggcagg acagcaaggg ggaggattgg
4801 gaagacaata gcaggcatgc tggggatgcg gtgggctcta tggcttctac tgggcggttt
4861 tatggacagc aagcgaaccg gaattgccag ctggggcgcc ctctggtaag gttgggaagc
4921 cctgcaaagt aaactggatg gctttcttgc cgccaaggat ctgatggcgc aggggatcaa
4981 gctctgatca agagacagga tgaggatcgt ttcgcatgat tgaacaagat ggattgcacg
5041 caggttctcc ggccgcttgg gtggagaggc tattcggcta tgactgggca caacagacaa
5101 tcggctgctc tgatgccgcc gtgttccggc tgtcagcgca ggggcgcccg gttctttttg
5161 tcaagaccga cctgtccggt gccctgaatg aactgcaaga cgaggcagcg cggctatcgt
5221 ggctggccac gacgggcgtt ccttgcgcag ctgtgctcga cgttgtcact gaagcgggaa
5281 gggactggct gctattgggc gaagtgccgg ggcaggatct cctgtcatct caccttgctc
5341 ctgccgagaa agtatccatc atggctgatg caatgcggcg gctgcatacg cttgatccgg
5401 ctacctgccc attcgaccac caagcgaaac atcgcatcga gcgagcacgt actcggatgg
5461 aagccggtct tgtcgatcag gatgatctgg acgaagagca tcaggggctc gcgccagccg
5521 aactgttcgc caggctcaag gcgagcatgc ccgacggcga ggatctcgtc gtgacccatg
5581 gcgatgcctg cttgccgaat atcatggtgg aaaatggccg cttttctgga ttcatcgact
5641 gtggccggct gggtgtggcg gaccgctatc aggacatagc gttggctacc cgtgatattg
5701 ctgaagagct tggcggcgaa tgggctgacc gcttcctcgt gctttacggt atcgccgctc
5761 ccgattcgca gcgcatcgcc ttctatcgcc ttcttgacga gttcttctga attattaacg
5821 cttacaattt cctgatgcgg tattttctcc ttacgcatct gtgcggtatt tcacaccgca
5881 tcaggtggca cttttcgggg aaatgtgcgc ggaaccccta tttgtttatt tttctaaata
5941 cattcaaata tgtatccgct catgagacaa taaccctgat aaatgcttca ataatagcac
6001 gtgctaaaac ttcattttta atttaaaagg atctaggtga agatcctttt tgataatctc
6061 atgaccaaaa tcccttaacg tgagttttcg ttccactgag cgtcagaccc cgtagaaaag
6121 atcaaaggat cttcttgaga tccttttttt ctgcgcgtaa tctgctgctt gcaaacaaaa
6181 aaaccaccgc taccagcggt ggtttgtttg ccggatcaag agctaccaac tctttttccg
6241 aaggtaactg gcttcagcag agcgcagata ccaaatactg ttcttctagt gtagccgtag
6301 ttaggccacc acttcaagaa ctctgtagca ccgcctacat acctcgctct gctaatcctg
6361 ttaccagtgg ctgctgccag tggcgataag tcgtgtctta ccgggttgga ctcaagacga
6421 tagttaccgg ataaggcgca gcggtcgggc tgaacggggg gttcgtgcac acagcccagc
6481 ttggagcgaa cgacctacac cgaactgaga tacctacagc gtgagctatg agaaagcgcc
6541 acgcttcccg aagggagaaa ggcggacagg tatccggtaa gcggcagggt cggaacagga
6601 gagcgcacga gggagcttcc agggggaaac gcctggtatc tttatagtcc tgtcgggttt
6661 cgccacctct gacttgagcg tcgatttttg tgatgctcgt caggggggcg gagcctatgg
6721 aaaaacgcca gcaacgcggc ctttttacgg ttcctggcct tttgctggcc ttttgctcac
6781 atgttctt

### SEQ ID NO: 4 SARS-CoV-2 Consensus Spike Antigen Amino Acid Insert Sequence of pGX9501 (IgE Leader sEquence Underlined)

1 MDWTWILFLV AAATRVHSSQ CVNLTTRTQL PPAYTNSFTR GVYYPDKVFR SSVLHSTQDL
61 FLPFFSNVTW FHAIHVSGTN GTKRFDNPVL PFNDGVYFAS TEKSNIIRGW IFGTTLDSKT
121 QSLLIVNNAT NVVIKVCEFQ FCNDPFLGVY YHKNNKSWME SEFRVYSSAN NCTFEYVSQP
181 FLMDLEGKQG NFKNLREFVF KNIDGYFKIY SKHTPINLVR DLPQGFSALE PLVDLPIGIN
241 ITRFQTLLAL HRSYLTPGDS SSGWTAGAAA YYVGYLQPRT FLLKYNENGT ITDAVDCALD
301 PLSETKCTLK SFTVEKGIYQ TSNFRVQPTE SIVRFPNITN LCPFGEVFNA TRFASVYAWN
361 RKRISNCVAD YSVLYNSASF STFKCYGVSP TKLNDLCFTN VYADSFVIRG DEVRQIAPGQ
421 TGKIADYNYK LPDDFTGCVI AWNSNNLDSK VGGNYNYLYR LFRKSNLKPF ERDISTEIYQ
481 AGSTPCNGVE GFNCYFPLQS YGFQPTNGVG YQPYRVVVLS FELLHAPATV CGPKKSTNLV
541 KNKCVNFNFN GLTGTGVLTE SNKKFLPFQQ FGRDIADTTD AVRDPQTLEI LDITPCSFGG
601 VSVITPGTNT SNQVAVLYQD VNCTEVPVAI HADQLTPTWR VYSTGSNVFQ TRAGCLIGAE
661 HVNNSYECDI PIGAGICASY QTQTNSPRRA RSVASQSIIA YTMSLGAENS VAYSNNSIAI
721 PTNFTISVTT EILPVSMTKT SVDCTMYICG DSTECSNLLL QYGSFCTQLN RALTGIAVEQ
781 DKNTQEVFAQ VKQIYKTPPI KDFGGFNFSQ ILPDPSKPSK RSFIEDLLFN KVTLADAGFI
841 KQYGDCLGDI AARDLICAQK FNGLTVLPPL LTDEMIAQYT SALLAGTITS GWTFGAGAAL
901 QIPFAMQMAY RFNGIGVTQN VLYENQKLIA NQFNSAIGKI QDSLSSTASA LGKLQDVVNQ
961 NAQALNTLVK QLSSNFGAIS SVLNDILSRL DKVEAEVQID RLITGRLQSL QTYVTQQLIR
1021 AAEIRASANL AATKMSECVL GQSKRVDFCG KGYHLMSFPQ SAPHGVVFLH VTYVPAQEKN
1081 FTTAPAICHD GKAHFPREGV FVSNGTHWFV TQRNFYEPQI ITTDNTFVSG NCDVVIGIVN
1141 NTVYDPLQPE LDSFKEELDK YFKNHTSPDV DLGDISGINA SVVNIQKEID RLNEVAKNLN
1201 ESLIDLQELG KYEQYIKWPW YIWLGFIAGL IAIVMVTIML CCMTSCCSCL KGCCSCGSCC
1261 KFDEDDSEPV LKGVKLHYT

### SEQ ID NO 5 DNA Insert Sequence of pGX9501 (IgE Leader Sequence Underlined)

1 ATGGATTGGA CTTGGATTCT CTTTCTCGTT GCTGCAGCCA CACGCGTTCA TAGCAGCCAG
61 TGTGTGAACC TGACCACCAG AACACAGCTG CCTCCTGCCT ACACCAACAG CTTCACCAGA
121 GGAGTCTACT ACCCAGACAA AGTCTTCAGA AGCTCTGTGC TGCACAGCAC CCAGGACCTG
181 TTCCTGCCTT TCTTCAGCAA CGTGACCTGG TTCCACGCCA TCCACGTGTC TGGCACCAAC
241 GGCACCAAGA GATTTGACAA CCCTGTTCTT CCTTTCAATG ATGGCGTGTA CTTTGCCAGC
301 ACAGAGAAGA GCAACATCAT CCGAGGCTGG ATCTTTGGCA CCACCCTGGA CAGCAAAACC
361 CAGAGCCTGC TGATCGTGAA CAACGCCACC AACGTGGTCA TCAAGGTGTG TGAGTTCCAG
421 TTCTGCAATG ACCCTTTCCT GGGCGTGTAC TACCACAAGA ACAACAAGTC CTGGATGGAG
481 TCTGAGTTCA GAGTCTACAG CTCTGCCAAC AACTGCACAT TTGAATATGT GTCCCAGCCT
541 TTCCTGATGG ACCTGGAGGG CAAGCAGGGC AACTTTAAGA ACCTGAGAGA ATTTGTGTTC
601 AAGAACATCG ATGGCTACTT CAAGATCTAC AGCAAGCACA CACCCATCAA CCTGGTGAGA
661 GACCTGCCTC AGGGCTTCTC TGCCCTGGAG CCTCTGGTGG ACCTGCCCAT CGGCATCAAC
721 ATCACCAGAT TCCAGACCCT GCTGGCCCTG CACAGAAGCT ACCTGACCCC AGGAGACAGC
781 AGCAGCGGCT GGACAGCTGG AGCTGCTGCC TACTACGTGG GCTACCTGCA GCCCAGGACC
841 TTCCTGCTGA AGTACAACGA AAATGGCACC ATCACAGATG CTGTTGACTG TGCCCTGGAC
901 CCTCTTAGCG AGACCAAGTG CACCCTGAAG TCCTTCACAG TGGAGAAAGG CATCTACCAG
961 ACCAGCAACT TCCGAGTGCA GCCAACAGAG AGCATCGTGA GATTTCCAAA CATCACCAAC
1021 CTGTGCCCTT TTGGAGAAGT CTTCAATGCC ACCAGATTTG CTTCTGTGTA CGCCTGGAAC
1081 AGAAAAAGAA TCAGCAACTG TGTGGCTGAC TACTCTGTGC TGTACAACTC TGCCTCCTTC
1141 TCCACCTTCA AGTGCTATGG AGTCTCTCCA ACCAAGCTGA ATGACCTGTG CTTCACCAAC
1201 GTGTATGCTG ACAGCTTTGT GATCAGAGGA GATGAAGTGC GGCAGATTGC TCCTGGCCAG
1261 ACAGGCAAGA TTGCTGACTA CAACTACAAG CTGCCTGATG ACTTCACAGG CTGTGTCATC
1321 GCCTGGAACA GCAACAACCT GGACAGCAAG GTGGGCGGCA ACTACAACTA CCTGTACAGA
1381 CTTTTCAGGA AGAGCAACCT GAAGCCTTTT GAAAGAGACA TCTCCACAGA GATCTACCAG
1441 GCTGGCAGCA CACCCTGCAA TGGTGTGGAA GGCTTCAACT GCTACTTCCC TCTGCAGAGC
1501 TACGGCTTCC AGCCAACAAA TGGCGTGGGC TACCAGCCTT ACAGAGTGGT GGTGCTGTCC
1561 TTTGAGCTGC TGCACGCCCC TGCCACAGTG TGTGGCCCCA AGAAGAGCAC CAACCTGGTG
1621 AAGAACAAAT GTGTGAACTT CAATTTCAAT GGCCTGACAG GCACAGGAGT GCTGACAGAG
1681 AGCAACAAGA AGTTTCTTCC TTTCCAGCAG TTTGGAAGAG ACATTGCTGA CACCACAGAT
1741 GCTGTGAGAG ATCCTCAGAC CCTGGAGATC CTGGATATCA CACCCTGCTC CTTTGGAGGA
1801 GTTTCTGTCA TCACACCTGG CACCAATACC AGCAACCAAG TGGCTGTGCT GTACCAAGAT
1861 GTGAATTGCA CAGAAGTGCC TGTGGCCATC CACGCTGACC AGCTGACACC CACCTGGAGA
1921 GTGTACAGCA CAGGCAGCAA TGTTTTCCAG ACAAGAGCTG GCTGCCTGAT TGGAGCAGAG
1981 CACGTGAACA ACAGCTATGA ATGTGACATC CCTATTGGAG CTGGCATCTG TGCCAGCTAC
2041 CAGACCCAAA CCAACAGCCC AAGAAGAGCC AGATCTGTGG CCAGCCAGAG CATCATCGCC
2101 TACACCATGA GCCTGGGAGC TGAGAACTCT GTGGCCTACA GCAACAACAG CATCGCCATC
2161 CCCACCAACT TCACCATCTC TGTGACCACA GAGATCCTGC CTGTGTCCAT GACCAAGACA
2221 TCTGTGGACT GCACCATGTA CATCTGTGGA GACAGCACAG AATGCAGCAA CCTGCTGCTG
2281 CAGTACGGCT CCTTCTGCAC CCAGCTGAAC AGAGCCCTGA CAGGCATCGC TGTGGAGCAG
2341 GACAAGAACA CACAGGAAGT GTTTGCCCAG GTGAAGCAGA TCTACAAAAC ACCACCCATC
2401 AAGGACTTTG GAGGCTTCAA TTTCTCCCAA ATCCTGCCTG ACCCCAGCAA GCCTTCCAAG
2461 AGAAGCTTCA TTGAAGACCT GCTGTTCAAC AAAGTGACCC TGGCTGATGC TGGCTTCATC
2521 AAGCAGTATG GAGACTGCCT GGGAGACATT GCTGCCAGAG ACCTGATCTG TGCCCAGAAG
2581 TTTAATGGCC TGACTGTGCT GCCTCCTCTG CTGACAGATG AAATGATCGC CCAGTACACA
2641 TCTGCCCTGC TGGCTGGCAC CATCACCAGT GGCTGGACAT TTGGAGCTGG AGCTGCCCTG
2701 CAGATCCCTT TTGCCATGCA GATGGCCTAC AGATTTAATG GCATCGGCGT GACCCAGAAC
2761 GTGCTGTACG AGAACCAGAA GCTGATCGCC AACCAGTTCA ACTCTGCCAT CGGCAAGATC
2821 CAGGACAGCC TGAGCAGCAC AGCCTCTGCC CTGGGCAAGC TGCAGGATGT GGTGAACCAA
2881 AACGCCCAGG CCCTGAACAC CCTGGTGAAG CAGCTGAGCA GCAACTTTGG AGCCATCTCC
2941 TCTGTGCTGA ATGACATCCT GAGCCGGCTG GACAAGGTGG AAGCAGAAGT GCAGATCGAC
3001 AGACTCATCA CAGGCCGCCT GCAGAGCCTG CAGACCTACG TGACCCAGCA GCTGATCAGA
3061 GCTGCTGAGA TCCGGGCCTC TGCCAACCTG GCTGCCACCA AGATGTCAGA ATGTGTGCTG
3121 GGCCAGAGCA AAAGAGTGGA CTTCTGTGGC AAAGGCTACC ACCTGATGTC CTTCCCTCAG
3181 TCTGCTCCTC ACGGCGTGGT GTTCCTGCAC GTGACCTACG TGCCTGCCCA GGAGAAGAAC
3241 TTCACCACAG CTCCTGCCAT CTGCCACGAT GGCAAGGCCC ACTTCCCAAG AGAAGGTGTC
3301 TTTGTGTCCA ATGGCACCCA CTGGTTCGTG ACCCAGAGAA ACTTCTACGA GCCTCAGATC
3361 ATCACCACAG ACAACACATT TGTGTCTGGC AACTGTGATG TGGTCATCGG CATCGTGAAC
3421 AACACAGTTT ATGACCCTCT GCAGCCTGAG CTGGACAGCT TCAAAGAAGA GCTGGACAAG
3481 TACTTCAAGA ACCACACATC TCCAGATGTG GACCTGGGAG ACATCTCTGG CATCAATGCC
3541 TCTGTGGTGA ACATCCAGAA GGAAATTGAC AGGCTGAACG AAGTGGCCAA GAACCTGAAC
3601 GAAAGCCTCA TCGACCTGCA GGAGCTGGGC AAGTACGAGC AGTACATCAA GTGGCCTTGG
3661 TACATCTGGC TGGGCTTCAT CGCTGGCCTC ATCGCCATCG TGATGGTGAC CATCATGCTG
3721 TGCTGCATGA CCAGCTGCTG CTCTTGCCTG AAGGGCTGCT GCAGCTGTGG CAGCTGCTGC
3781 AAGTTTGATG AAGATGACTC TGAGCCTGTG CTGAAGGGCG TGAAGCTGCA CTACACA

### SEQ ID NO 6 Single Strand DNA Sequence of pGX9501

1 gctgcttcgc gatgtacggg ccagatatac gcgttgacat tgattattga ctagttatta
61 atagtaatca attacggggt cattagttca tagcccatat atggagttcc gcgttacata
121 acttacggta aatggcccgc ctggctgacc gcccaacgac ccccgcccat tgacgtcaat
181 aatgacgtat gttcccatag taacgccaat agggactttc cattgacgtc aatgggtgga
241 gtatttacgg taaactgccc acttggcagt acatcaagtg tatcatatgc caagtacgcc
301 ccctattgac gtcaatgacg gtaaatggcc cgcctggcat tatgcccagt acatgacctt
361 atgggacttt cctacttggc agtacatcta cgtattagtc atcgctatta ccatggtgat
421 gcggttttgg cagtacatca atgggcgtgg atagcggttt gactcacggg gatttccaag
481 tctccacccc attgacgtca atgggagttt gttttggcac caaaatcaac gggactttcc
541 aaaatgtcgt aacaactccg ccccattgac gcaaatgggc ggtaggcgtg tacggtggga
601 ggtctatata agcagagctc tctggctaac tagagaaccc actgcttact ggcttatcga
661 aattaatacg actcactata gggagaccca agctggctag cgtttaaact taagcttggt
721 accgagctcg gatccgccac catggattgg acttggattc tctttctcgt tgctgcagcc
781 acacgcgttc atagcagcca gtgtgtgaac ctgaccacca gaacacagct gcctcctgcc
841 tacaccaaca gcttcaccag aggagtctac tacccagaca aagtcttcag aagctctgtg
901 ctgcacagca cccaggacct gttcctgcct ttcttcagca acgtgacctg gttccacgcc
961 atccacgtgt ctggcaccaa cggcaccaag agatttgaca accctgttct tcctttcaat
1021 gatggcgtgt actttgccag cacagagaag agcaacatca tccgaggctg gatctttggc
1081 accaccctgg acagcaaaac ccagagcctg ctgatcgtga acaacgccac caacgtggtc
1141 atcaaggtgt gtgagttcca gttctgcaat gaccctttcc tgggcgtgta ctaccacaag
1201 aacaacaagt cctggatgga gtctgagttc agagtctaca gctctgccaa caactgcaca
1261 tttgaatatg tgtcccagcc tttcctgatg gacctggagg gcaagcaggg caactttaag
1321 aacctgagag aatttgtgtt caagaacatc gatggctact tcaagatcta cagcaagcac
1381 acacccatca acctggtgag agacctgcct cagggcttct ctgccctgga gcctctggtg
1441 gacctgccca tcggcatcaa catcaccaga ttccagaccc tgctggccct gcacagaagc
1501 tacctgaccc caggagacag cagcagcggc tggacagctg gagctgctgc ctactacgtg
1561 ggctacctgc agcccaggac cttcctgctg aagtacaacg aaaatggcac catcacagat
1621 gctgttgact gtgccctgga ccctcttagc gagaccaagt gcaccctgaa gtccttcaca
1681 gtggagaaag gcatctacca gaccagcaac ttccgagtgc agccaacaga gagcatcgtg
1741 agatttccaa acatcaccaa cctgtgccct tttggagaag tcttcaatgc caccagattt
1801 gcttctgtgt acgcctggaa cagaaaaaga atcagcaact gtgtggctga ctactctgtg
1861 ctgtacaact ctgcctcctt ctccaccttc aagtgctatg gagtctctcc aaccaagctg
1921 aatgacctgt gcttcaccaa cgtgtatgct gacagctttg tgatcagagg agatgaagtg
1981 cggcagattg ctcctggcca gacaggcaag attgctgact acaactacaa gctgcctgat
2041 gacttcacag gctgtgtcat cgcctggaac agcaacaacc tggacagcaa ggtgggcggc
2101 aactacaact acctgtacag acttttcagg aagagcaacc tgaagccttt tgaaagagac
2161 atctccacag agatctacca ggctggcagc acaccctgca atggtgtgga aggcttcaac
2221 tgctacttcc ctctgcagag ctacggcttc cagccaacaa atggcgtggg ctaccagcct
2281 tacagagtgg tggtgctgtc ctttgagctg ctgcacgccc ctgccacagt gtgtggcccc
2341 aagaagagca ccaacctggt gaagaacaaa tgtgtgaact tcaatttcaa tggcctgaca
2401 ggcacaggag tgctgacaga gagcaacaag aagtttcttc ctttccagca gtttggaaga
2461 gacattgctg acaccacaga tgctgtgaga gatcctcaga ccctggagat cctggatatc
2521 acaccctgct cctttggagg agtttctgtc atcacacctg gcaccaatac cagcaaccaa
2581 gtggctgtgc tgtaccaaga tgtgaattgc acagaagtgc ctgtggccat ccacgctgac
2641 cagctgacac ccacctggag agtgtacagc acaggcagca atgttttcca gacaagagct
2701 ggctgcctga ttggagcaga gcacgtgaac aacagctatg aatgtgacat ccctattgga
2761 gctggcatct gtgccagcta ccagacccaa accaacagcc caagaagagc cagatctgtg
2821 gccagccaga gcatcatcgc ctacaccatg agcctgggag ctgagaactc tgtggcctac
2881 agcaacaaca gcatcgccat ccccaccaac ttcaccatct ctgtgaccac agagatcctg
2941 cctgtgtcca tgaccaagac atctgtggac tgcaccatgt acatctgtgg agacagcaca
3001 gaatgcagca acctgctgct gcagtacggc tccttctgca cccagctgaa cagagccctg
3061 acaggcatcg ctgtggagca ggacaagaac acacaggaag tgtttgccca ggtgaagcag
3121 atctacaaaa caccacccat caaggacttt ggaggcttca atttctccca aatcctgcct
3181 gaccccagca agccttccaa gagaagcttc attgaagacc tgctgttcaa caaagtgacc
3241 ctggctgatg ctggcttcat caagcagtat ggagactgcc tgggagacat tgctgccaga
3301 gacctgatct gtgcccagaa gtttaatggc ctgactgtgc tgcctcctct gctgacagat
3361 gaaatgatcg cccagtacac atctgccctg ctggctggca ccatcaccag tggctggaca
3421 tttggagctg gagctgccct gcagatccct tttgccatgc agatggccta cagatttaat
3481 ggcatcggcg tgacccagaa cgtgctgtac gagaaccaga agctgatcgc caaccagttc
3541 aactctgcca tcggcaagat ccaggacagc ctgagcagca cagcctctgc cctgggcaag
3601 ctgcaggatg tggtgaacca aaacgcccag gccctgaaca ccctggtgaa gcagctgagc
3661 agcaactttg gagccatctc ctctgtgctg aatgacatcc tgagccggct ggacaaggtg
3721 gaagcagaag tgcagatcga cagactcatc acaggccgcc tgcagagcct gcagacctac
3781 gtgacccagc agctgatcag agctgctgag atccgggcct ctgccaacct ggctgccacc
3841 aagatgtcag aatgtgtgct gggccagagc aaaagagtgg acttctgtgg caaaggctac
3901 cacctgatgt ccttccctca gtctgctcct cacggcgtgg tgttcctgca cgtgacctac
3961 gtgcctgccc aggagaagaa cttcaccaca gctcctgcca tctgccacga tggcaaggcc
4021 cacttcccaa gagaaggtgt ctttgtgtcc aatggcaccc actggttcgt gacccagaga
4081 aacttctacg agcctcagat catcaccaca gacaacacat ttgtgtctgg caactgtgat
4141 gtggtcatcg gcatcgtgaa caacacagtt tatgaccctc tgcagcctga gctggacagc
4201 ttcaaagaag agctggacaa gtacttcaag aaccacacat ctccagatgt ggacctggga
4261 gacatctctg gcatcaatgc ctctgtggtg aacatccaga aggaaattga caggctgaac
4321 gaagtggcca agaacctgaa cgaaagcctc atcgacctgc aggagctggg caagtacgag
4381 cagtacatca agtggccttg gtacatctgg ctgggcttca tcgctggcct catcgccatc
4441 gtgatggtga ccatcatgct gtgctgcatg accagctgct gctcttgcct gaagggctgc
4501 tgcagctgtg gcagctgctg caagtttgat gaagatgact ctgagcctgt gctgaagggc
4561 gtgaagctgc actacacatg ataactcgag tctagagggc ccgtttaaac ccgctgatca
4621 gcctcgactg tgccttctag ttgccagcca tctgttgttt gcccctcccc cgtgccttcc
4681 ttgaccctgg aaggtgccac tcccactgtc ctttcctaat aaaatgagga aattgcatcg
4741 cattgtctga gtaggtgtca ttctattctg gggggtgggg tggggcagga cagcaagggg
4801 gaggattggg aagacaatag caggcatgct ggggatgcgg tgggctctat ggcttctact
4861 gggcggtttt atggacagca agcgaaccgg aattgccagc tggggcgccc tctggtaagg
4921 ttgggaagcc ctgcaaagta aactggatgg ctttcttgcc gccaaggatc tgatggcgca
4981 ggggatcaag ctctgatcaa gagacaggat gaggatcgtt tcgcatgatt gaacaagatg
5041 gattgcacgc aggttctccg gccgcttggg tggagaggct attcggctat gactgggcac
5101 aacagacaat cggctgctct gatgccgccg tgttccggct gtcagcgcag gggcgcccgg
5161 ttctttttgt caagaccgac ctgtccggtg ccctgaatga actgcaagac gaggcagcgc
5221 ggctatcgtg gctggccacg acgggcgttc cttgcgcagc tgtgctcgac gttgtcactg
5281 aagcgggaag ggactggctg ctattgggcg aagtgccggg gcaggatctc ctgtcatctc
5341 accttgctcc tgccgagaaa gtatccatca tggctgatgc aatgcggcgg ctgcatacgc
5401 ttgatccggc tacctgccca ttcgaccacc aagcgaaaca tcgcatcgag cgagcacgta
5461 ctcggatgga agccggtctt gtcgatcagg atgatctgga cgaagagcat caggggctcg
5521 cgccagccga actgttcgcc aggctcaagg cgagcatgcc cgacggcgag gatctcgtcg
5581 tgacccatgg cgatgcctgc ttgccgaata tcatggtgga aaatggccgc ttttctggat
5641 tcatcgactg tggccggctg ggtgtggcgg accgctatca ggacatagcg ttggctaccc
5701 gtgatattgc tgaagagctt ggcggcgaat gggctgaccg cttcctcgtg ctttacggta
5761 tcgccgctcc cgattcgcag cgcatcgcct tctatcgcct tcttgacgag ttcttctgaa
5821 ttattaacgc ttacaatttc ctgatgcggt attttctcct tacgcatctg tgcggtattt
5881 cacaccgcat caggtggcac ttttcgggga aatgtgcgcg gaacccctat ttgtttattt
5941 ttctaaatac attcaaatat gtatccgctc atgagacaat aaccctgata aatgcttcaa
6001 taatagcacg tgctaaaact tcatttttaa tttaaaagga tctaggtgaa gatccttttt
6061 gataatctca tgaccaaaat cccttaacgt gagttttcgt tccactgagc gtcagacccc
6121 gtagaaaaga tcaaaggatc ttcttgagat cctttttttc tgcgcgtaat ctgctgcttg
6181 caaacaaaaa aaccaccgct accagcggtg gtttgtttgc cggatcaaga gctaccaact
6241 ctttttccga aggtaactgg cttcagcaga gcgcagatac caaatactgt tcttctagtg
6301 tagccgtagt taggccacca cttcaagaac tctgtagcac cgcctacata cctcgctctg
6361 ctaatcctgt taccagtggc tgctgccagt ggcgataagt cgtgtcttac cgggttggac
6421 tcaagacgat agttaccgga taaggcgcag cggtcgggct gaacgggggg ttcgtgcaca
6481 cagcccagct tggagcgaac gacctacacc gaactgagat acctacagcg tgagctatga
6541 gaaagcgcca cgcttcccga agggagaaag gcggacaggt atccggtaag cggcagggtc
6601 ggaacaggag agcgcacgag ggagcttcca gggggaaacg cctggtatct ttatagtcct
6661 gtcgggtttc gccacctctg acttgagcgt cgatttttgt gatgctcgtc aggggggcgg
6721 agcctatgga aaaacgccag caacgcggcc tttttacggt tcctggcctt ttgctggcct
6781 tttgctcaca tgttctt

### SEQ ID NO: 7 SARS-CoV-2 Consensus Spike Antigen Amino Acid Insert Sequence of pGX9527 (IgE Leader Sequence Underlined)

1 MDWTWILFLV AAATRVHSSQ CVNFTTRTQL PPAYTNSFTR GVYYPDKVFR SSVLHSTQDL
61 FLPFFSNVTW FHAISGTNGT KRFDNPVLPF NDGVYFASTE KSNIIRGWIF GTTLDSKTQS
121 LLIVNNATNV VIKVCEFQFC NDPFLGVYYH KNNKSWMESE FRVYSSANNC TFEYVSQPFL
181 MDLEGKQGNF KNLREFVFKN IDGYFKIYSK HTPINLVRDL PQGFSVLEPL VDLPIGINIT
241 RFQTLLALHR SYLTPGDSSS GWTAGAAAYY VGYLQPRTFL LKYNENGTIT DAVDCALDPL
301 SETKCTLKSF TVEKGIYQTS NFRVQPTESI VRFPNITNLC PFGEVFNATR FASVYAWNRK
361 RISNCVADYS VLYNSASFST FKCYGVSPTK LNDLCFTNVY ADSFVIRGDE VRQIAPGQTG
421 NIADYNYKLP DDFTGCVIAW NSNNLDSKVG GNYNYLYRLF RKSNLKPFER DISTEIYQAG
481 STPCNGVKGF NCYFPLQSYG FQPTYGVGYQ PYRVVVLSFE LLHAPATVCG PKKSTNLVKN
541 KCVNFNFNGL TGTGVLTESN KKFLPFQQFG RDIADTTDAV RDPQTLEILD ITPCSFGGVS
601 VITPGTNTSN QVAVLYQGVN CTEVPVAIHA DQLTPTWRVY STGSNVFQTR AGCLIGAEHV
661 NNSYECDIPI GAGICASYQT QTNSHRRARS VASQSIIAYT MSLGAENSVA YSNNSIAIPT
721 NFTISVTTEI LPVSMTKTSV DCTMYICGDS TECSNLLLQY GSFCTQLNRA LTGIAVEQDK
781 NTQEVFAQVK QIYKTPPIKD FGGFNFSQIL PDPSKPSKRS FIEDLLFNKV TLADAGFIKQ
841 YGDCLGDIAA RDLICAQKFN GLTVLPPLLT DEMIAQYTSA LLAGTITSGW TFGAGAALQI
901 PFAMQMAYRF NGIGVTQNVL YENQKLIANQ FNSAIGKIQD SLSSTASALG KLQDVVNQNA
961 QALNTLVKQL SSNFGAISSV LNDILSRLDP PEAEVQIDRL ITGRLQSLQT YVTQQLIRAA
1021 EIRASANLAA TKMSECVLGQ SKRVDFCGKG YHLMSFPQSA PHGVVFLHVT YVPAQEKNFT
1081 TAPAICHDGK AHFPREGVFV SNGTHWFVTQ RNFYEPQIIT TDNTFVSGNC DVVIGIVNNT
1141 VYDPLQPELD SFKEELDKYF KNHTSPDVDL GDISGINASV VNIQKEIDRL NEVAKNLNES
1201 LIDLQELGKY EQYIKWPWYI WLGFIAGLIA IVMVTIMLCC MTSCCSCLKG CCSCGSCCKF
1261 DEDDSEPVLK GVKLHYT

### SEQ ID NO: 8 Nucleic Acid Sequence Encoding SARS-CoV-2 Consensus Spike Antigen Amino Acid Insert Sequence of pGX9527 (IgE Leader Sequence Underlined)

atggattggacttggattctctttctcgttgctgcagccacacgcgttca tagcagccagtgtgtgaacttcaccaccagaacacagctgcctcctgcct
acaccaacagcttcaccagaggagtctactacccagacaaagtcttcaga
agctctgtgctgcacagcacccaggacctgttcctgcctttcttcagcaa
cgtgacctggttccacgccatctctggcaccaacggcaccaagagatttg
acaaccctgttcttcctttcaatgatggcgtgtactttgccagcacagag
aagagcaacatcatccgaggctggatctttggcaccaccctggacagcaa
aacccagagcctgctgatcgtgaacaacgccaccaacgtggtcatcaagg
tgtgtgagttccagttctgcaatgaccctttcctgggcgtgtactaccac
aagaacaacaagtcctggatggagtctgagttcagagtctacagctctgc
caacaactgcacatttgaatatgtgtcccagcctttcctgatggacctgg
agggcaagcagggcaactttaagaacctgagagaatttgtgttcaagaac
atcgatggctacttcaagatctacagcaagcacacacccatcaacctggt
gagagacctgcctcagggcttctctgtgctggagcctctggtggacctgc
ccatcggcatcaacatcaccagattccagaccctgctggccctgcacaga
agctacctgaccccaggagacagcagcagcggctggacagctggagctgc
tgcctactacgtgggctacctgcagcccaggaccttcctgctgaagtaca
acgaaaatggcaccatcacagatgctgttgactgtgccctggaccctctt
agcgagaccaagtgcaccctgaagtccttcacagtggagaaaggcatcta
ccagaccagcaacttccgagtgcagccaacagagagcatcgtgagatttc
caaacatcaccaacctgtgcccttttggagaagtcttcaatgccaccaga
tttgcttctgtgtacgcctggaacagaaaaagaatcagcaactgtgtggc
tgactactctgtgctgtacaactctgcctccttctccaccttcaagtgct
atggagtctctccaaccaagctgaatgacctgtgcttcaccaacgtgtat
gctgacagctttgtgatcagaggagatgaagtgcggcagattgctcctgg
ccagacaggcaacattgctgactacaactacaagctgcctgatgacttca
caggctgtgtcatcgcctggaacagcaacaacctggacagcaaggtgggc
ggcaactacaactacctgtacagacttttcaggaagagcaacctgaagcc
ttttgaaagagacatctccacagagatctaccaggctggcagcacaccct
gcaatggtgtgaagggcttcaactgctacttccctctgcagagctacggc
ttccagccaacatatggcgtgggctaccagccttacagagtggtggtgct
gtcctttgagctgctgcacgcccctgccacagtgtgtggccccaagaaga
gcaccaacctggtgaagaacaaatgtgtgaacttcaatttcaatggcctg
acaggcacaggagtgctgacagagagcaacaagaagtttcttcctttcca
gcagtttggaagagacattgctgacaccacagatgctgtgagagatcctc
agaccctggagatcctggatatcacaccctgctcctttggaggagtttct
gtcatcacacctggcaccaataccagcaaccaagtggctgtgctgtacca
aggagtgaattgcacagaagtgcctgtggccatccacgctgaccagctga
cacccacctggagagtgtacagcacaggcagcaatgttttccagacaaga
gctggctgcctgattggagcagagcacgtgaacaacagctatgaatgtga
catccctattggagctggcatctgtgccagctaccagacccaaaccaaca
gccacagaagagccagatctgtggccagccagagcatcatcgcctacacc
atgagcctgggagctgagaactctgtggcctacagcaacaacagcatcgc
catccccaccaacttcaccatctctgtgaccacagagatcctgcctgtgt
ccatgaccaagacatctgtggactgcaccatgtacatctgtggagacagc
acagaatgcagcaacctgctgctgcagtacggctccttctgcacccagct
gaacagagccctgacaggcatcgctgtggagcaggacaagaacacacagg
aagtgtttgcccaggtgaagcagatctacaaaacaccacccatcaaggac
tttggaggcttcaatttctcccaaatcctgcctgaccccagcaagccttc
caagagaagcttcattgaagacctgctgttcaacaaagtgaccctggctg
atgctggcttcatcaagcagtatggagactgcctgggagacattgctgcc
agagacctgatctgtgcccagaagtttaatggcctgactgtgctgcctcc
tctgctgacagatgaaatgatcgcccagtacacatctgccctgctggctg
gcaccatcaccagtggctggacatttggagctggagctgccctgcagatc
ccttttgccatgcagatggcctacagatttaatggcatcggcgtgaccca
gaacgtgctgtacgagaaccagaagctgatcgccaaccagttcaactctg
ccatcggcaagatccaggacagcctgagcagcacagcctctgccctgggc
aagctgcaggatgtggtgaaccaaaacgcccaggccctgaacaccctggt
gaagcagctgagcagcaactttggagccatctcctctgtgctgaatgaca
tcctgagccggctggaccctccagaagcagaagtgcagatcgacagactc
atcacaggccgcctgcagagcctgcagacctacgtgacccagcagctgat
cagagctgctgagatccgggcctctgccaacctggctgccaccaagatgt
cagaatgtgtgctgggccagagcaaaagagtggacttctgtggcaaaggc
taccacctgatgtccttccctcagtctgctcctcacggcgtggtgttcct
gcacgtgacctacgtgcctgcccaggagaagaacttcaccacagctcctg
ccatctgccacgatggcaaggcccacttcccaagagaaggtgtctttgtg
tccaatggcacccactggttcgtgacccagagaaacttctacgagcctca
gatcatcaccacagacaacacatttgtgtctggcaactgtgatgtggtca
tcggcatcgtgaacaacacagtttatgaccctctgcagcctgagctggac
agcttcaaagaagagctggacaagtacttcaagaaccacacatctccaga
tgtggacctgggagacatctctggcatcaatgcctctgtggtgaacatcc
agaaggaaattgacaggctgaacgaagtggccaagaacctgaacgaaagc
ctcatcgacctgcaggagctgggcaagtacgagcagtacatcaagtggcc
ttggtacatctggctgggcttcatcgctggcctcatcgccatcgtgatgg
tgaccatcatgctgtgctgcatgaccagctgctgctcttgcctgaagggc
tgctgcagctgtggcagctgctgcaagtttgatgaagatgactctgagcc
tgtgctgaagggcgtgaagctgcactacacatgataa

### SEQ ID NO: 9 Single Strand DNA Sequence of pGX9527

1 gctgcttcgc gatgtacggg ccagatatac gcgttgacat tgattattga ctagttatta
61 atagtaatca attacggggt cattagttca tagcccatat atggagttcc gcgttacata
121 acttacggta aatggcccgc ctggctgacc gcccaacgac ccccgcccat tgacgtcaat
181 aatgacgtat gttcccatag taacgccaat agggactttc cattgacgtc aatgggtgga
241 gtatttacgg taaactgccc acttggcagt acatcaagtg tatcatatgc caagtacgcc
301 ccctattgac gtcaatgacg gtaaatggcc cgcctggcat tatgcccagt acatgacctt
361 atgggacttt cctacttggc agtacatcta cgtattagtc atcgctatta ccatggtgat
421 gcggttttgg cagtacatca atgggcgtgg atagcggttt gactcacggg gatttccaag
481 tctccacccc attgacgtca atgggagttt gttttggcac caaaatcaac gggactttcc
541 aaaatgtcgt aacaactccg ccccattgac gcaaatgggc ggtaggcgtg tacggtggga
601 ggtctatata agcagagctc tctggctaac tagagaaccc actgcttact ggcttatcga
661 aattaatacg actcactata gggagaccca agctggctag cgtttaaact taagcttggt
721 accgagctcg gatccgccac catggattgg acttggattc tctttctcgt tgctgcagcc
781 acacgcgttc atagcagcca gtgtgtgaac ttcaccacca gaacacagct gcctcctgcc
841 tacaccaaca gcttcaccag aggagtctac tacccagaca aagtcttcag aagctctgtg
901 ctgcacagca cccaggacct gttcctgcct ttcttcagca acgtgacctg gttccacgcc
961 atctctggca ccaacggcac caagagattt gacaaccctg ttcttccttt caatgatggc
1021 gtgtactttg ccagcacaga gaagagcaac atcatccgag gctggatctt tggcaccacc
1081 ctggacagca aaacccagag cctgctgatc gtgaacaacg ccaccaacgt ggtcatcaag
1141 gtgtgtgagt tccagttctg caatgaccct ttcctgggcg tgtactacca caagaacaac
1201 aagtcctgga tggagtctga gttcagagtc tacagctctg ccaacaactg cacatttgaa
1261 tatgtgtccc agcctttcct gatggacctg gagggcaagc agggcaactt taagaacctg
1321 agagaatttg tgttcaagaa catcgatggc tacttcaaga tctacagcaa gcacacaccc
1381 atcaacctgg tgagagacct gcctcagggc ttctctgtgc tggagcctct ggtggacctg
1441 cccatcggca tcaacatcac cagattccag accctgctgg ccctgcacag aagctacctg
1501 accccaggag acagcagcag cggctggaca gctggagctg ctgcctacta cgtgggctac
1561 ctgcagccca ggaccttcct gctgaagtac aacgaaaatg gcaccatcac agatgctgtt
1621 gactgtgccc tggaccctct tagcgagacc aagtgcaccc tgaagtcctt cacagtggag
1681 aaaggcatct accagaccag caacttccga gtgcagccaa cagagagcat cgtgagattt
1741 ccaaacatca ccaacctgtg cccttttgga gaagtcttca atgccaccag atttgcttct
1801 gtgtacgcct ggaacagaaa aagaatcagc aactgtgtgg ctgactactc tgtgctgtac
1861 aactctgcct ccttctccac cttcaagtgc tatggagtct ctccaaccaa gctgaatgac
1921 ctgtgcttca ccaacgtgta tgctgacagc tttgtgatca gaggagatga agtgcggcag
1981 attgctcctg gccagacagg caacattgct gactacaact acaagctgcc tgatgacttc
2041 acaggctgtg tcatcgcctg gaacagcaac aacctggaca gcaaggtggg cggcaactac
2101 aactacctgt acagactttt caggaagagc aacctgaagc cttttgaaag agacatctcc
2161 acagagatct accaggctgg cagcacaccc tgcaatggtg tgaagggctt caactgctac
2221 ttccctctgc agagctacgg cttccagcca acatatggcg tgggctacca gccttacaga
2281 gtggtggtgc tgtcctttga gctgctgcac gcccctgcca cagtgtgtgg ccccaagaag
2341 agcaccaacc tggtgaagaa caaatgtgtg aacttcaatt tcaatggcct gacaggcaca
2401 ggagtgctga cagagagcaa caagaagttt cttcctttcc agcagtttgg aagagacatt
2461 gctgacacca cagatgctgt gagagatcct cagaccctgg agatcctgga tatcacaccc
2521 tgctcctttg gaggagtttc tgtcatcaca cctggcacca ataccagcaa ccaagtggct
2581 gtgctgtacc aaggagtgaa ttgcacagaa gtgcctgtgg ccatccacgc tgaccagctg
2641 acacccacct ggagagtgta cagcacaggc agcaatgttt tccagacaag agctggctgc
2701 ctgattggag cagagcacgt gaacaacagc tatgaatgtg acatccctat tggagctggc
2761 atctgtgcca gctaccagac ccaaaccaac agccacagaa gagccagatc tgtggccagc
2821 cagagcatca tcgcctacac catgagcctg ggagctgaga actctgtggc ctacagcaac
2881 aacagcatcg ccatccccac caacttcacc atctctgtga ccacagagat cctgcctgtg
2941 tccatgacca agacatctgt ggactgcacc atgtacatct gtggagacag cacagaatgc
3001 agcaacctgc tgctgcagta cggctccttc tgcacccagc tgaacagagc cctgacaggc
3061 atcgctgtgg agcaggacaa gaacacacag gaagtgtttg cccaggtgaa gcagatctac
3121 aaaacaccac ccatcaagga ctttggaggc ttcaatttct cccaaatcct gcctgacccc
3181 agcaagcctt ccaagagaag cttcattgaa gacctgctgt tcaacaaagt gaccctggct
3241 gatgctggct tcatcaagca gtatggagac tgcctgggag acattgctgc cagagacctg
3301 atctgtgccc agaagtttaa tggcctgact gtgctgcctc ctctgctgac agatgaaatg
3361 atcgcccagt acacatctgc cctgctggct ggcaccatca ccagtggctg gacatttgga
3421 gctggagctg ccctgcagat cccttttgcc atgcagatgg cctacagatt taatggcatc
3481 ggcgtgaccc agaacgtgct gtacgagaac cagaagctga tcgccaacca gttcaactct
3541 gccatcggca agatccagga cagcctgagc agcacagcct ctgccctggg caagctgcag
3601 gatgtggtga accaaaacgc ccaggccctg aacaccctgg tgaagcagct gagcagcaac
3661 tttggagcca tctcctctgt gctgaatgac atcctgagcc ggctggaccc tccagaagca
3721 gaagtgcaga tcgacagact catcacaggc cgcctgcaga gcctgcagac ctacgtgacc
3781 cagcagctga tcagagctgc tgagatccgg gcctctgcca acctggctgc caccaagatg
3841 tcagaatgtg tgctgggcca gagcaaaaga gtggacttct gtggcaaagg ctaccacctg
3901 atgtccttcc ctcagtctgc tcctcacggc gtggtgttcc tgcacgtgac ctacgtgcct
3961 gcccaggaga agaacttcac cacagctcct gccatctgcc acgatggcaa ggcccacttc
4021 ccaagagaag gtgtctttgt gtccaatggc acccactggt tcgtgaccca gagaaacttc
4081 tacgagcctc agatcatcac cacagacaac acatttgtgt ctggcaactg tgatgtggtc
4141 atcggcatcg tgaacaacac agtttatgac cctctgcagc ctgagctgga cagcttcaaa
4201 gaagagctgg acaagtactt caagaaccac acatctccag atgtggacct gggagacatc
4261 tctggcatca atgcctctgt ggtgaacatc cagaaggaaa ttgacaggct gaacgaagtg
4321 gccaagaacc tgaacgaaag cctcatcgac ctgcaggagc tgggcaagta cgagcagtac
4381 atcaagtggc cttggtacat ctggctgggc ttcatcgctg gcctcatcgc catcgtgatg
4441 gtgaccatca tgctgtgctg catgaccagc tgctgctctt gcctgaaggg ctgctgcagc
4501 tgtggcagct gctgcaagtt tgatgaagat gactctgagc ctgtgctgaa gggcgtgaag
4561 ctgcactaca catgataact cgagtctaga gggcccgttt aaacccgctg atcagcctcg
4621 actgtgcctt ctagttgcca gccatctgtt gtttgcccct cccccgtgcc ttccttgacc
4681 ctggaaggtg ccactcccac tgtcctttcc taataaaatg aggaaattgc atcgcattgt
4741 ctgagtaggt gtcattctat tctggggggt ggggtggggc aggacagcaa gggggaggat
4801 tgggaagaca atagcaggca tgctggggat gcggtgggct ctatggcttc tactgggcgg
4861 ttttatggac agcaagcgaa ccggaattgc cagctggggc gccctctggt aaggttggga
4921 agccctgcaa agtaaactgg atggctttct tgccgccaag gatctgatgg cgcaggggat
4981 caagctctga tcaagagaca ggatgaggat cgtttcgcat gattgaacaa gatggattgc
5041 acgcaggttc tccggccgct tgggtggaga ggctattcgg ctatgactgg gcacaacaga
5101 caatcggctg ctctgatgcc gccgtgttcc ggctgtcagc gcaggggcgc ccggttcttt
5161 ttgtcaagac cgacctgtcc ggtgccctga atgaactgca agacgaggca gcgcggctat
5221 cgtggctggc cacgacgggc gttccttgcg cagctgtgct cgacgttgtc actgaagcgg
5281 gaagggactg gctgctattg ggcgaagtgc cggggcagga tctcctgtca tctcaccttg
5341 ctcctgccga gaaagtatcc atcatggctg atgcaatgcg gcggctgcat acgcttgatc
5401 cggctacctg cccattcgac caccaagcga aacatcgcat cgagcgagca cgtactcgga
5461 tggaagccgg tcttgtcgat caggatgatc tggacgaaga gcatcagggg ctcgcgccag
5521 ccgaactgtt cgccaggctc aaggcgagca tgcccgacgg cgaggatctc gtcgtgaccc
5581 atggcgatgc ctgcttgccg aatatcatgg tggaaaatgg ccgcttttct ggattcatcg
5641 actgtggccg gctgggtgtg gcggaccgct atcaggacat agcgttggct acccgtgata
5701 ttgctgaaga gcttggcggc gaatgggctg accgcttcct cgtgctttac ggtatcgccg
5761 ctcccgattc gcagcgcatc gccttctatc gccttcttga cgagttcttc tgaattatta
5821 acgcttacaa tttcctgatg cggtattttc tccttacgca tctgtgcggt atttcacacc
5881 gcatcaggtg gcacttttcg gggaaatgtg cgcggaaccc ctatttgttt atttttctaa
5941 atacattcaa atatgtatcc gctcatgaga caataaccct gataaatgct tcaataatag
6001 cacgtgctaa aacttcattt ttaatttaaa aggatctagg tgaagatcct ttttgataat
6061 ctcatgacca aaatccctta acgtgagttt tcgttccact gagcgtcaga ccccgtagaa
6121 aagatcaaag gatcttcttg agatcctttt tttctgcgcg taatctgctg cttgcaaaca
6181 aaaaaaccac cgctaccagc ggtggtttgt ttgccggatc aagagctacc aactcttttt
6241 ccgaaggtaa ctggcttcag cagagcgcag ataccaaata ctgttcttct agtgtagccg
6301 tagttaggcc accacttcaa gaactctgta gcaccgccta catacctcgc tctgctaatc
6361 ctgttaccag tggctgctgc cagtggcgat aagtcgtgtc ttaccgggtt ggactcaaga
6421 cgatagttac cggataaggc gcagcggtcg ggctgaacgg ggggttcgtg cacacagccc
6481 agcttggagc gaacgaccta caccgaactg agatacctac agcgtgagct atgagaaagc
6541 gccacgcttc ccgaagggag aaaggcggac aggtatccgg taagcggcag ggtcggaaca
6601 ggagagcgca cgagggagct tccaggggga aacgcctggt atctttatag tcctgtcggg
6661 tttcgccacc tctgacttga gcgtcgattt ttgtgatgct cgtcaggggg gcggagccta
6721 tggaaaaacg ccagcaacgc ggccttttta cggttcctgg ccttttgctg gccttttgct
6781 cacatgttct t

What is claimed:

1. A nucleic acid molecule encoding a Severe Acute Respiratory Syndrome coronavirus 2 (SARS-CoV-2) spike antigen, wherein:
the nucleic acid molecule encodes amino acids 19 to 1276 of SEQ ID NO: 1
the nucleic acid molecule encodes the amino acid sequence of SEQ ID NO: 1;
the nucleic acid molecule comprises the nucleic acid sequence of nucleotides 55 to 3828 of SEQ ID NO: 2;
the nucleic acid molecule comprises the nucleic acid sequence of SEQ ID NO: 2; or
the nucleic acid molecule comprises the nucleic acid sequence of SEQ ID NO: 3.

2. An expression vector comprising the nucleic acid molecule according to claim 1.

3. An immunogenic composition comprising an effective amount of the expression vector according to claim 2 and a pharmaceutically acceptable excipient.

4. The immunogenic composition according to claim 3 wherein the pharmaceutically acceptable excipient comprises a buffer.

5. The immunogenic composition according to claim 4 wherein the buffer is a saline-sodium citrate buffer.

6. The immunogenic composition of claim 5, wherein the composition comprises 10 mg of the expression vector per milliliter of saline-sodium citrate buffer.

7. The immunogenic composition according to claim 3, further comprising an adjuvant.

8. A method of inducing an immune response against Severe Acute Respiratory Syndrome coronavirus 2 (SARS-CoV-2) in a subject in need thereof, the method comprising administering to the subject an effective amount of an immunogenic composition comprising:
an expression vector comprising a nucleic acid molecule encoding a Severe Acute Respiratory Syndrome coronavirus 2 (SARS-CoV-2) spike antigen, wherein:
the nucleic acid molecule encodes amino acids 19 to 1276 of SEQ ID NO: 1 the nucleic acid molecule encodes the amino acid sequence of SEQ ID NO: 1;
the nucleic acid molecule comprises the nucleic acid sequence of nucleotides 55 to 3828 of SEQ ID NO: 2;
the nucleic acid molecule comprises the nucleic acid sequence of SEQ ID NO: 2; or
the nucleic acid molecule comprises the nucleic acid sequence of SEQ ID NO: 3,
and a pharmaceutically acceptable excipient.

9. The method of claim 8, wherein administering comprises at least one of electroporation and injection.

10. The method of claim 8, wherein administering comprises parenteral administration followed by electroporation.

11. The method of claim 8, wherein an initial dose of about 0.5 mg to about 2.0 mg of the vector is administered to the subject.

12. The method of claim 11, wherein the initial dose is 0.5 mg, 1.0 mg or 2.0 mg of the vector.

13. The method of claim 11, wherein a subsequent dose of about 0.5 mg to about 2.0 mg of the vector is administered to the subject about four weeks after the initial dose.

14. The method of claim 13, wherein the subsequent dose is 0.5 mg, 1.0 mg, or 2.0 mg of the vector.

15. The method of claim 13, wherein one or more further subsequent doses of about 0.5 mg to about 2.0 mg of the vector is administered to the subject at least twelve weeks after the initial dose.

16. The method of claim 8, wherein the immunogenic composition comprises pGX9541, INO-4803 drug product or a biosimilar thereof.

17. The method of claim 8, further comprising administering to the subject at least one additional agent for the treatment of SARS-CoV-2 infection or the treatment or prevention of a disease or disorder associated with SARS-CoV-2 infection.

18. The method of claim 17, wherein the at least one additional agent comprises a SARS-CoV-2 wild-type matched vaccine, pGX9501, INO-4800 drug product or a biosimilar thereof, pGX9527, or INO-4802 drug product or a biosimilar thereof.

19. The method of claim 17 wherein the immunogenic composition is administered to the subject before, concurrently with, or after the additional agent.

20. A method of protecting a subject in need thereof from infection with Severe Acute Respiratory Syndrome coronavirus 2 (SARS-CoV-2), the method comprising administering to the subject an effective amount of an immunogenic composition comprising:
an expression vector comprising a nucleic acid molecule encoding a Severe Acute Respiratory Syndrome coronavirus 2 (SARS-CoV-2) spike antigen, wherein:
the nucleic acid molecule encodes amino acids 19 to 1276 of SEQ ID NO: 1 the nucleic acid molecule encodes the amino acid sequence of SEQ ID NO: 1;
the nucleic acid molecule comprises the nucleic acid sequence of nucleotides 55 to 3828 of SEQ ID NO: 2;
the nucleic acid molecule comprises the nucleic acid sequence of SEQ ID NO: 2; or
the nucleic acid molecule comprises the nucleic acid sequence of SEQ ID NO: 3,
and a pharmaceutically acceptable excipient.

21. The method of claim 20, wherein administering comprises at least one of electroporation and injection.

22. The method of claim 20, wherein administering comprises parenteral administration followed by electroporation.

23. The method of claim 20, wherein an initial dose of about 0.5 mg to about 2.0 mg of the vector is administered to the subject.

24. The method of claim 23, wherein the initial dose is 0.5 mg, 1.0 mg or 2.0 mg of the vector.

25. The method of claim 23, wherein a subsequent dose of about 0.5 mg to about 2.0 mg of the vector is administered to the subject about four weeks after the initial dose.

26. The method of claim 25, wherein the subsequent dose is 0.5 mg, 1.0 mg or 2.0 mg of the vector.

27. The method of claim 25, wherein one or more further subsequent doses of about 0.5 mg to about 2.0 mg of the vector is administered to the subject at least twelve weeks after the initial dose.

28. The method of claim 20, wherein the immunogenic composition comprises pGX9541, INO-4803 drug product or a biosimilar thereof.

29. The method of claim 20, further comprising administering to the subject at least one additional agent for the treatment of SARS-CoV-2 infection or the treatment or prevention of a disease or disorder associated with SARS-CoV-2 infection.

30. The method of claim 29, wherein the at least one additional agent comprises a SARS-CoV-2 wild-type matched vaccine, pGX9501, INO-4800 drug product or a biosimilar thereof, pGX9527, or INO-4802 drug product or a biosimilar thereof.

31. The method of claim 20, wherein the immunogenic composition is administered to the subject before, concurrently with, or after the additional agent.

32. A method of treating a subject in need thereof against Severe Acute Respiratory Syndrome coronavirus 2 (SARS-CoV-2) infection, the method comprising administering to the subject an effective amount of an immunogenic composition comprising:
an expression vector comprising a nucleic acid molecule encoding a Severe Acute Respiratory Syndrome coronavirus 2 (SARS-CoV-2) spike antigen, wherein:
the nucleic acid molecule encodes amino acids 19 to 1276 of SEQ ID NO: 1 the nucleic acid molecule encodes the amino acid sequence of SEQ ID NO: 1;
the nucleic acid molecule comprises the nucleic acid sequence of nucleotides 55 to 3828 of SEQ ID NO: 2;
the nucleic acid molecule comprises the nucleic acid sequence of SEQ ID NO: 2; or
the nucleic acid molecule comprises the nucleic acid sequence of SEQ ID NO: 3,
and a pharmaceutically acceptable excipient,
wherein the subject is thereby resistant to one or more SARS-CoV-2 strains.

33. The method of claim 32, wherein administering comprises at least one of electroporation and injection.

34. The method of claim 32, wherein administering comprises parenteral administration followed by electroporation.

35. The method of claim 32, wherein an initial dose of about 0.5 mg to about 2.0 mg of the vector is administered to the subject.

36. The method of claim 35, wherein the initial dose is 0.5 mg, 1.0 mg or 2.0 mg of the vector.

37. The method of claim 35, wherein a subsequent dose of about 0.5 mg to about 2.0 mg of the vector is administered to the subject about four weeks after the initial dose.

38. The method of claim 37, wherein the subsequent dose is 0.5 mg, 1.0 mg or 2.0 mg of the vector.

39. The method of claim 37, wherein one or more further subsequent doses of about 0.5 mg to about 2.0 mg of the vector is administered to the subject at least twelve weeks after the initial dose.

40. The method of claim 32, wherein the immunogenic composition comprises pGX9541, INO-4803 drug product or a biosimilar thereof.

41. The method of claim 32, further comprising administering to the subject at least one additional agent for the treatment of SARS-CoV-2 infection or the treatment or prevention of a disease or disorder associated with SARS-CoV-2 infection.

42. The method of claim 41, wherein the at least one additional agent comprises a SARS-CoV-2 wild-type matched vaccine, pGX9501, INO-4800 drug product or a biosimilar thereof, pGX9527, or INO-4802 drug product or a biosimilar thereof.

43. The method of claim 32, wherein the immunogenic composition is administered to the subject before, concurrently with, or after the additional agent.

44. A Severe Acute Respiratory Syndrome coronavirus 2 (SARS-CoV-2) spike antigen comprising amino acids 19 to 1276 of SEQ ID NO: 1 or the amino acid sequence of SEQ ID NO: 1.
